(19) United States
(12) Patent Application Publication
(10) Pub. No.: US 2017/0291942 A1
(43) Pub. Date: Oct. 12, 2017

(54) Treatment of Depression Using Agents that Block Binding of IL-6 to IL-6 Receptor

(71) Applicant: Janssen Biotech, Inc., Horsham, PA (US)

(72) Inventors: Guang Chen, San Diego, CA (US); Yu Sun, Belle Meade, NJ (US); Dai Wang, Bridgewater, NJ (US); Gayle Wittenberg, Plainsboro, NJ (US)

(21) Appl. No.: 15/480,924

(22) Filed: Apr. 6, 2017

Related U.S. Application Data
(60) Provisional application No. 62/319,558, filed on Apr. 7, 2016.

(51) Int. Cl. C07K 16/24

(52) U.S. Cl. CPC C07K 16/248; C07K 2317/565; C07K 2317/56; C07K 2317/76; C07K 2317/21; C07K 2317/24; A61K 2039/505

(57) ABSTRACT

A method for treating depression or fatigue in a subject comprises administering an agent that blocks binding of IL-6 to IL-6 receptor, for example, an anti-IL-6 antibody or an antigen-binding fragment thereof that may comprise a heavy chain variable region and a light chain variable region of SEQ ID NO:99 and SEQ ID NO:97, respectively or a heavy chain variable region and a light chain variable region of SEQ ID NO:139 and SEQ ID NO:140, respectively. In addition, a method for determining responsiveness of an individual having depression to treatment with IL-6 antibody or antigen-binding fragment thereof, comprises: (a) measuring the amount of soluble IL-6 receptor (sIL-6R) in a biological sample from the subject; (b) providing a threshold value correlating the amount of sIL-6R and responsiveness; (c) comparing the amount of sIL-6R to the threshold value to determine responsiveness; and (d) treating the individual with an agent that blocks binding of IL-6 to IL-6 receptor.

### CROSS-REFERENCE TO RELATED APPLICATIONS

This application claims the benefit of priority of U.S. Provisional Application No. 62/319,558, filed 7 Apr. 2016, the entire contents of which are incorporated herein by reference in their entireties.

### FIELD OF THE INVENTION

The invention relates to a method of treating depression or fatigue. More particularly, the invention provides compositions and methods for treating depression or fatigue using agents that block binding of IL-6 to IL-6 receptor, specifically using anti-IL-6 antibodies. The invention also provides a method for determining responsiveness of an individual having depression to the treatment with IL-6 antibody.

### BACKGROUND OF THE INVENTION

Interleukin-6 (IL-6) is a pro-inflammatory cytokine that is produced by many different cell types. In vivo, stimulated monocytes, fibroblasts, and endothelial cells represent the main sources of IL-6. Other cells such as macrophages, T and B lymphocytes, granulocytes, keratinocytes, mast cells, osteoblasts, chrondrocytes, glial cells, and smooth muscle cells also produce IL-6 after stimulation (Kishimoto, T., Blood 74:1-10 (1989) and Kurihara, N. et al., J. Immunology 144:4226-4230 (1990)). Several tumor cells also produce IL-6 (Smith, P. C. et al. Cytokine and Growth Factor Reviews 12:33-40 (2001)); IL-6 was indicated to be a prognostic factor for prostate cancer progression (Nakashima, J. et al. Clinical Cancer Research 6:2702-2706 (2000)). IL-6 production can be regulated by IL-6 itself and depending upon cell type, IL-6 can stimulate or inhibit its own synthesis.

IL-6 can bind to the IL-6 receptor expressed on mitogen-activated B cells, T cells, peripheral monocytes, and certain tumors (Ishimi, Y. et al., J. Immunology 145:3297-3303 (1990)). The IL-6 receptor has at least two different components and is composed of an alpha chain called gp80 that is responsible for IL-6 binding and a beta chain designated gp130 that is needed for signal transduction (Adebanjo, O. et al., J. Cell Biology 142:1347-1356 (1998) and Poli, V. et al., EMBO 13:1189-1196 (1994)). The cytokine family which includes IL-6, LIF, Oncostatin M, IL-11, CNTF, and CT-1 all signal through gp130 after binding to their cognate receptors. In addition, all members of the IL-6 cytokine family can induce hepatic expression of acute phase proteins (Bellido, T. et al., J. Clin. Investigation 97:431-437 (1996)).

There are at least two major biological functions of IL-6: mediation of acute phase proteins and acting as a differentiation and activation factor (Avvisti, G. et al., Baillieres Clinical Hematology 8:815-829 (1995) and Poli, V. et al., EMBO 13:1189-1196 (1994)). Acute phase proteins are known to regulate immune responses, mediate inflammation, and play a role in tissue remodeling. As a differentiation and activation factor, IL-6 induces B cells to differentiate and secrete antibody, it induces T cells to differentiate into cytotoxic T cells, activates cell signaling factors, and promotes hematopoiesis (Ishimi, Y. et al., J. Immunology 145:3297-3303 (1990)). IL-6 is prominently involved in many critical bodily functions and processes. As a result, physiological processes including bone metabolism, neoplastic transformation, and immune and inflammatory responses can be enhanced, suppressed, or prevented by manipulation of the biological activity of IL-6 in vivo by means of an antibody (Adebanjo, O. et al., J. Cell Biology 142:1347-1356 (1998)).

IL-6 is also implicated in the regulation of synaptic plasticity, neuronal development and survival, and neurogenesis (Gruol, D. L., Neuropharmacology, 96:42-54 (2015)). It can be produced both centrally, by microglia, astrocytes, endothelial cells and certain neurons, and peripherally, released from T-cells and macrophages (Gruol, D. L., Neuropharmacology, 96:42-54 (2015)). Evidence suggests that peripheral IL-6 can enter the brain to exert central effects (Banks, W. A., et al., Neurosci Lett 179:53-56 (1994)). IL-6 activates two forms of signaling: cis signaling, mediated by the membrane bound IL-6 receptor (IL-6R), and trans-signaling through the soluble IL-6 receptor (sIL-6R), by which IL-6 exerts a more widespread influence to cells which do not express the IL-6R (Hunter, C. A., et al., Nat Immunol 16:448-457 (2015)).

Elevation of IL-6, both peripherally and centrally, has been widely reported in patients with major depression disorder (MDD). Several clinical studies and meta-analysis have shown that IL-6 levels are significantly elevated in depressed patients, in plasma or serum (Maes, M., et al., J Affect Disord 34:301-309 (1995)). Some studies have reported an association between peripheral IL-6 elevation and the symptom severity and chronicity of depression episodes, and between CSF IL-6 elevation and suicidal ideation (O'Donovan, A., et al., Depress Anxiety 30:307-314 (2013)). Elevation of IL-6 mRNA levels is also found in leukocytes from depressed patients (Cattaneo, A., et al., Neuropsychopharmacology 38; 377-385 (2013)). Peripheral IL-6 elevation has been linked to central changes through an association with reductions in hippocampal volume in depressed patients (Frodl, T., et al., Transl Psychiatry 2:e88 (2012)). Elevated levels of sIL-6R have also been reported in depressed patients (Maes, M., et al., J Affect Disord 34:301-309 (1995)) and IL-6 trans-signaling, specifically, has been associated with depression and CNS dysfunction (Maes, M., et al., Expert Opin Ther Targets 18:495-512 (2014)).

Interferon alpha (IFN-α) treatment in patients treated for Hepatitis C frequently induces symptoms of depression and fatigue with a concomitant increase in IL-6 plasma (Bonaccorso, S., et al., Psychiatry Res 105:45-55 (2001)) and CSF (Raison, C. L., et al., Biol Psychiatry 65:296-303 (2009)) levels. A promoter polymorphism in the IL-6 gene results in lower IL-6 transcription and hepatitis C patients with this low efficiency IL-6 promoter show reduced risk of developing depressive symptoms when treated with IFN-α (Bull, S. J., et al., Mol Psychiatry 14:1095-1104 (2009)). These IFN-α data also support a role of IL-6 in depression.

Social and psychological stress is a key contributor and trigger of clinical depression. Elevations of IL-6 and its downstream effectors in the blood and brain have been reported in several animal stress paradigms of depression (Kinsey, S. G., et al., Physiol Behav 93:628-636 (2008)). The social defeat stress paradigm captures many facets of depression etiology, underlying neurobiology, behavioral deficits of clinical depression, and response to antidepressants (Berton, O., et al., Science 311:864-868 (2006)). After 10 days of social defeat stress section, 60-70% of mice (susceptible) develop social interaction deficits and anhedonia-like abnormalities (Golden, S. A., et al., Nat Protoc 6:1183-1191 (2006)). Susceptible mice display higher levels of IL-6 in the leukocytes prior to social defeat stress compared with resilient animals, with no difference observed in the brain regions studied (Hodes, G. E., et al., Proc Natl Acad Sci USA 111:16136-16141 (2014)). Peripheral IL-6 injection alone does not induce depression-like deficits; however, it increases the likelihood of being susceptible to a subthreshold social defeat paradigm. Furthermore, transplanting hematopoietic progenitor cells from the bone marrow of stress-susceptible mice increases IL-6 production and social avoidance behavior after stress exposure in the bone marrow chimers. IL-6 knockout mice, and mice treated with anti-IL-6 antibodies administered i.p. do not develop social interaction deficits (Hodes, G. E., et al., Proc Natl Acad Sci USA 111:16136-16141 (2014)). These preclinical data further support a role of peripheral IL-6 in depression and suggest peripheral IL-6 neutralization as a therapeutic approach to treat depression.

### SUMMARY OF THE INVENTION

The present invention provides a method for treating depression or fatigue in a subject comprising administering to the subject an effective amount of a pharmaceutical composition comprising an agent that blocks binding of IL-6 to IL-6 receptor. In another embodiment, the agent that blocks binding of IL-6 to IL-6 receptor comprises an isolated antibody or an antigen-binding fragment thereof. In another embodiment, the isolated antibody or an antigen-binding fragment thereof comprises a heavy chain variable region and a light chain variable region of SEQ ID NO:99 and SEQ ID NO:97, respectively [Sirukumab]. In another embodiment, the isolated antibody or an antigen-binding fragment thereof comprises a heavy chain variable region and a light chain variable region of SEQ ID NO:139 and SEQ ID NO:140, respectively [Siltuximab].

The present invention also provides a method for determining responsiveness of an individual having depression to the treatment with IL-6 antibody or a fragment thereof, comprising: (a) measuring the amount of soluble IL-6 receptor (sIL-6R) in a biological sample from the subject; (b) providing a threshold value correlating the amount of sIL-6R and responsiveness; (c) comparing the amount of sIL-6R to the threshold value; wherein responsiveness is determined when the amount of sIL-6R exceeds the threshold value and/or wherein a lack of responsiveness is determined when the amount of sIL-6R does not exceed the threshold value; and (d) treating the individual with an agent that blocks binding of IL-6 to IL-6 receptor.

One embodiment of the invention is a method for treating depression or fatigue in a subject comprising administering to the subject an effective amount of a pharmaceutical composition comprising an agent that blocks binding of IL-6 to IL-6 receptor.

In another embodiment, the subject has rheumatoid arthritis.

In another embodiment, the subject has multicentric Castleman's disease.

In another embodiment, the agent that blocks binding of IL-6 to IL-6 receptor comprises an isolated antibody or an antigen-binding fragment thereof.

In another embodiment, the isolated antibody or an antigen-binding fragment thereof comprises a heavy chain variable regions and a light chain variable regions of SEQ ID NO:99 and SEQ ID NO:97 respectively.

In another embodiment, the isolated antibody or an antigen-binding fragment thereof comprises a heavy chain variable region and a light chain variable regions of SEQ ID NO:139 and SEQ ID NO:140 respectively.

In another embodiment, the isolated antibody or an antigen-binding fragment thereof is administered at a dose of about 25-100 mg about every 2-4 weeks.

In another embodiment, the isolated antibody or an antigen-binding fragment thereof is administered at a dose selected from the group comprising about 100 mg every 2 weeks, about 25 mg every 4 weeks, about 50 mg every 4 weeks, and about 100 mg every 4 weeks.

In another embodiment, the isolated antibody or an antigen-binding fragment thereof is administered at a dose of about 11 mg/kg every 3 weeks.

In another embodiment, the isolated antibody or an antigen-binding fragment thereof is administered subcutaneously.

In another embodiment, the isolated antibody or an antigen-binding fragment thereof is administered intravenously.

Another embodiment of the invention is a method for determining responsiveness of an individual having depression to the treatment with IL-6 antibody or a fragment thereof, comprising:

a. measuring the amount of soluble IL-6 receptor (sIL-6R) in a biological sample from the subject;
b. providing a threshold value correlating the amount of sIL-6R and responsiveness; and
c. comparing the amount of sIL-6R to the threshold value; wherein responsiveness is determined when the amount of sIL-6R exceeds the threshold value and/or wherein a lack of responsiveness is determined when the amount of sIL-6R does not exceed the threshold value.

In another embodiment, the threshold value is about 45 ng/mL.

In another embodiment, the biological sample is serum.

In another embodiment, the patient has rheumatoid arthritis or multicentric Castleman's disease.

In another embodiment, the IL-6 antibody or a fragment thereof comprises a heavy chain variable regions and a light chain variable regions of SEQ ID NO:99 and SEQ ID NO:97 respectively.

In another embodiment, the IL-6 antibody or a fragment thereof comprises a heavy chain variable regions and a light chain variable regions of SEQ ID NO:139 and SEQ ID NO:140 respectively.

### DESCRIPTION OF THE FIGURES

FIG. 1A: shows the distribution and symptom characteristics of patients with (filled bar) and without (open bar) PDMA at trial entry in a sirukumab cohort.

FIG. 1B: shows the distribution and symptom characteristics of patients with (filled bar) and without (open bar) PDMA at trial entry in a siltuximab cohort.

FIG. 2A: shows depression total score pre- (open bar) and post-(filled bar) treatment in patients with and without PDMA treated with sirukumab for 12 weeks. Left panel: not adjusted for RA severity. Right panel: adjusted for RA severity.

FIG. 2B: shows depression total score pre- and post-treatment in patients with and without PDMA treated with Siltuximab for 6 weeks. Left panel: not adjusted for MCD severity. Right panel: adjusted for MCD severity.

FIG. 3A: shows fatigue total score pre- (open bar) and post-(filled bar) treatment in patients with and without PDMA treated with sirukumab for 12 weeks. Left panel: not adjusted for RA severity. Right panel: adjusted for RA severity.

FIG. 3B: shows fatigue total score pre- and post-treatment in patients with and without PDMA treated with Siltuximab for 6 weeks. Left panel: not adjusted for MCD severity. Right panel: adjusted for MCD severity.

FIG. 4A: shows depression total score pre- (open bar) and post-(filled bar) treatment in patients with PDMA who were RA responders or non-responders as defined by the ACR50 criteria. Patients were stratified by baseline sIL-6R levels

FIG. 4B: shows pre- and post-treatment depression total scores in patients treated with sirukumab or placebo stratified by baseline IL-6R>=45 ng/mL.

FIG. 4C shows depression total score pre- and post-treatment in patients with PDMA who were MCD responders or non-responders, stratified by baseline sIL-6R levels

FIG. 5A: shows Fatigue total score pre- (open bar) and post-(filled bar) treatment in patients with PDMA at the entry point who were RA responders or non-responders as defined by the ACR50 criteria.

FIG. 5B: shows Fatigue total score pre- (open bar) and post-(filled bar) treatment in RA patients treated with sirukumab or placebo with PDMA at the entry point stratified by baseline IL-6R>=45 ng/mL.

FIG. 5C: shows Fatigue total score pre- (open bar) and post-(filled bar) treatment in patients with PDMA at the entry point who were MCD responders or non-responders as defined by the ACR50 criteria.

### DETAILED DESCRIPTION OF THE INVENTION

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats.

The disclosed subject matter may be understood more readily by reference to the following detailed description taken in connection with the accompanying figures, which form a part of this disclosure. It is to be understood that the disclosed subject matter are not limited to those described and/or shown herein, and that the terminology used herein is for the purpose of describing particular embodiments by way of example only and is not intended to be limiting of the claimed subject matter.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

### Definitions

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

“Treating” or “treatment” refer to any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters, including the results of a physical examination, neurological examination, or psychiatric evaluations.

“Depression”, also known as “unipolar affective disorder”, is characterized by a combination of symptoms such as lowered mood, loss of energy, loss of interest, feeling of physical illness, poor concentration, altered appetite, altered sleep and a slowing down of physical and mental functions resulting in a relentless feeling of hopelessness, helplessness, guilt, and anxiety.

“Fatigue” refers to a condition of physical and/or mental exhaustion. Fatigue can be subjectively described as feeling weary, tired, exhausted, malaise, listless, lack of energy, or feeling run down.

“Effective amount” or “therapeutically effective amount” refers to an amount effective, at dosages and for periods of time necessary, to achieve a desired therapeutic result. A therapeutically effective amount of an agent that blocks binding of IL-6 to IL-6 receptor may vary according to factors such as the disease state, age, sex, and weight of the individual, and the ability of the antibody to elicit a desired response in the individual. A therapeutically effective amount is also one in which any toxic or detrimental effects of the agent are outweighed by the therapeutically beneficial effects.

As used herein, an “anti-IL-6 antibody,” “IL-6 antibody,” “anti-IL-6 antibody portion,” or “anti-IL-6 antibody fragment” and/or “anti-IL-6 antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-6 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-6 activity or binding, or with IL-6 receptor activity or binding, in vitro, in situ and/or in vivo. As a non-limiting example, a suitable anti-IL-6 antibody, specified portion or variant of the present invention can bind at least one IL-6 molecule, or specified portions, variants or domains thereof. A suitable anti-IL-6 antibody, specified portion, or variant can also optionally affect at least one of IL-6 activity or function, such as but not limited to, RNA, DNA or protein synthesis, IL-6 release, IL-6 receptor signaling, membrane IL-6 cleavage, IL-6 activity, IL-6 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-6. For example, antibody fragments capable of binding to IL-6 or portions thereof, including, but not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

As used herein “chimeric” antibodies or “humanized” antibodies or “CDR-grafted” include any combination of the herein described murine CDR's with one or more proteins or peptides derived from a non-murine, preferably, human antibody. In accordance with the invention, chimeric or humanized antibodies are provided wherein the CDR's are derived from the murine CLB-8 antibody capable of binding human IL-6 and at least a portion, or the remainder of the antibody is derived from one or more human antibodies. Thus, the human part of the antibody may include the framework, CL, CH domains (e.g., CH1, CH2, CH3), hinge, (VL, VH)) regions which are substantially non-immunogenic in humans. The regions of the antibody that are derived from human antibodies need not have 100% identity with human antibodies. In a preferred embodiment, as many of the human amino acid residues as possible are retained in order for the immunogenicity to be negligible, but the human residues may be modified as necessary to support the antigen binding site formed by the CDR's while simultaneously maximizing the humanization of the antibody. Such changes or variations optionally and preferably retain or reduce the immunogenicity in humans or other species relative to non-modified antibodies.

It is pointed out that a humanized antibody can be produced by a non-human animal or prokaryotic or eukaryotic cell that is capable of expressing functionally rearranged human immunoglobulin (e.g., heavy chain and/or light chain) genes. Further, when the antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, the term “human engineered antibody” is an antibody with at least fully human frameworks and constant regions (CL, CH domains (e.g., CH1, CH2, CH3), and hinge), and CDRs derived from antigen binding antibodies. Fully human frameworks comprise frameworks that correspond to human germline sequences as well as sequences with somatic mutations. CDRs may be derived from one or more CDRs that bind to IL-6 in the context of any antibody framework. For example, the CDRs of the human engineered antibody of the present invention may be derived from CDRs that bind IL-6 in the context of a mouse antibody framework and then are engineered to bind IL-6 in the context of a fully human framework. Often, the human engineered antibody is substantially non-immunogenic in humans.

Anti-IL-6 antibodies useful in the methods and compositions of the present invention can optionally be characterized by high affinity binding to IL-6 and, optionally and preferably, as having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as the incidence of titrable levels of antibodies to the anti-IL-6 antibody in patients treated with anti-IL-6 antibody as occurring in less than 25% of patients treated, preferably, in less than 10% of patients treated with the recommended dose for the recommended course of therapy during the treatment period.

“Subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

### Antibodies of the Present Invention—Production and Generation

### Chimeric Antibodies

In accordance with the present invention, the anti-IL-6 cCLB-8 antibody comprises an antibody in which the variable region or CDRs are derived from the murine CLB-8 antibody capable of binding to and inhibiting the function of human IL-6 and the framework and constant regions of the antibody are derived from one or more human antibodies. The variable region or CDRs derived from the murine CLB-8 antibody preferably have from about 90% to about 100% identity with the variable region or CDRs of the murine CLB-8 antibody, although any and all modifications, including substitutions, insertions and deletions, are contemplated so long as the chimeric antibody maintains the ability to bind to and inhibit IL-6. The regions of the chimeric, humanized or CDR-grafted antibodies that are derived from human antibodies need not have 100% identity with the human antibodies. In a preferred embodiment, as many of the human amino acid residues as possible are retained in order than immunogenicity is negligible, but the human residues, in particular residues of the framework region, are substituted as required and as taught herein below in accordance with the present invention. Such modifications as disclosed herein are necessary to support the antigen binding site formed by the CDRs while simultaneously maximizing the humanization of the antibody.

The CLB-8 murine monoclonal antibody against human IL-6 is known in the art (Brakenhoff et al, J. Immunol 145:561 (1990)). The present invention discloses chimeric, humanized or CDR grafted antibodies derived from the CDR regions of the CLB-8 murine monoclonal antibody and methods for preparing such antibodies. Each of the heavy and light chain variable regions contain three CDRs that combine to form the antigen binding site. The three CDRs are surrounded by four framework (FR) regions that primarily function to support the CDRs. The sequences of the CDRs within the sequences of the variable regions of the heavy and light chains can be identified by computer-assisted alignment according to Kabat et al. (1987) in Sequences of Proteins of Immunological Interest, 4^{th} ed., United States Department of Health and Human Services, U.S. Government Printing Office, Washington, D.C., or by molecular modeling of the variable regions, for example utilizing the ENCAD program as described by Levitt (1983) J. Mol. Biol. 168:595.

In a preferred embodiment the CDRs are derived from murine monoclonal antibody CLB-8.

The Preferred Heavy Chain CDRs have the Following Sequences:

CDR1
(SEQ ID NO: 141)
SFAMS
CDR2
(SEQ ID NO: 142)
EISSGGSYTYYPDTVTG
CDR3
(SEQ ID NO: 143)
GLWGYYALDY

The Preferred Light Chain CDRs have the Following Sequences:

CDR1
(SEQ. ID NO: 144)
SASSSVSYMY
CDR2
(SEQ. ID NO: 145)
DTSNLAS
CDR3
(SEQ. ID NO: 146)
QQWSGYPYT

The sequences of the CDRs of the murine CLB-8 antibody, may be modified by insertions, substitutions and deletions to the extent that the CDR-grafted antibody maintains the ability to bind to and inhibit human 11-6. The ordinarily skilled artisan can ascertain the maintenance of this activity by performing the functional assays described herein below. The CDRs can have, for example, from about 50% to about 100% homology to the CDRs of SEQ ID NOS: 141-146. In a preferred embodiment the CDRs have from about 80% to about 100% homology to the CDRs of SEQ ID NOS: 141-146. In a more preferred embodiment the CDRs have from about 90% to about 100% homology to the CDRs of SEQ ID NOS: 141-146. In a most preferred embodiment the CDRs have from about 100% homology to the CDRs of SEQ ID NOS: 141-146.

Alternatively, the entire heavy chain variable region and light chain variable region of the murine CLB-8 antibody (SEQ ID NOS. 139 and 140) may be combined with the human constant and framework regions to form the chimeric cCLB-8 antibody of the present invention.

### The Preferred Heavy Chain Variable Region and Light Chain Variable Region of the Murine CLB-8 Antibody (SEQ. ID NOS. 139 and 140)

### Heavy Chain Variable Region (SEQ. ID NOS. 139):

| | |
|---|---|
| 1 | EVQLVESGGK LLKPGGSLKL SCAASGFTFS SFAMSWFRQS PEKRLEWVAE ISSGGSYTYY |
| 61 | PDTVTGRFTI SRDNAKNTLY LEMSSLRSED TAMYYCARGL WGYYALDYWG QGTSVTVSS |

### Light Chain Variable Region (SEQ. ID NOS. 140):

| | |
|---|---|
| 1 | QIVLIQSPAI MSASPGEKVT MTCSASSSVS YMYWYQQKPG SSPRLLIYDT SNLASGVPVR |
| 61 | FSGSGSGTSY SLTISRMEAE DAATYYCQQW SGYPYTFGGG TKLEIK |

Human genes which encode the constant (C) regions of the chimeric antibodies, fragments and regions of the present invention can be derived from a human fetal liver library, by known methods. Human C region genes can be derived from any human cell including those which express and produce human immunoglobulins. The human CH region can be derived from any of the known classes or isotypes of human H chains, including gamma, μ, α, δ, ε, and subtypes thereof, such as G1, G2, G3 and G4. Since the H chain isotype is responsible for the various effector functions of an antibody, the choice of CH region will be guided by the desired effector functions, such as complement fixation, or activity in antibody-dependent cellular cytotoxicity (ADCC). Preferably, the CH region is derived from gamma 1 (IgG1).

The human CL region can be derived from either human L chain isotype, kappa or lambda, preferably kappa.

Genes encoding human immunoglobulin C regions are obtained from human cells by standard cloning techniques (Sambrook, et al. (Molecular Cloning: A Laboratory Manual, 2nd Edition, Cold Spring Harbor Press, Cold Spring Harbor, N.Y. (1989) and Ausubel et al, eds. Current Protocols in Molecular Biology (1987-1993)). Human C region genes are readily available from known clones containing genes representing the two classes of L chains, the five classes of H chains and subclasses thereof. Chimeric antibody fragments, such as F(abl)2 and Fab, can be prepared by designing a chimeric H chain gene which is appropriately truncated. For example, a chimeric gene encoding an H chain portion of an F(abl)2 fragment would include DNA sequences encoding the CH1 domain and hinge region of the H chain, followed by a translational stop codon to yield the truncated molecule.

Generally, in one example, chimeric antibodies, fragments and regions of the present invention are produced by cloning DNA segments encoding the H and L chain antigen-binding regions of the CLB-8 anti-IL-6 specific antibody, and joining these DNA segments to DNA segments encoding CH and CL regions, respectively, to produce chimeric immunoglobulin-encoding genes.

Thus, in a preferred embodiment, a fused chimeric gene is created which comprises a first DNA segment that encodes at least the antigen-binding region of non-human origin, such as a functionally rearranged V region with joining (J) segment, linked to a second DNA segment encoding at least a part of a human C region.

The sequences of the variable regions of the murine CLB-8 antibody, may be modified by insertions, substitutions and deletions to the extent that the chimeric antibody maintains the ability to bind to and inhibit human IL-6. The ordinarily skilled artisan can ascertain the maintenance of this activity by performing the functional assays described herein below. The variable regions can have, for example, from about 50% to about 100% homology to the variable regions of SEQ ID NOS:139-140. In a preferred embodiment, the variable regions have from about 80% to about 100% homology to the variable regions of SEQ ID NOS: 139-140. In a more preferred embodiment the variable regions have from about 90% to about 100% homology to the variable regions of SEQ ID NOS: 139-140. In a most preferred embodiment the variable regions have from about 100% homology to the CDRs of SEQ ID NOS: 139-140.

For convenience, the numbering scheme of Kabat et al., has been adopted herein. Residues are designated by lower case numbers or hyphens as necessary to conform the present sequences to the standard Kabat numbered sequence.

In accordance with the present invention, in the case of a CDR-grafted or humanized antibody where the CDR region of the CLB-8 antibody is combined with a human region, residues may be retained in the FR region which are idiosyncratic to the parent antibody, e.g. CLB-8. Residues that have been demonstrated to be critical in the humanization of other antibodies may also be retained. The foregoing guidelines are followed to the extent necessary to support the antigen binding site formed by the CDRs while simultaneously maximizing the humanization of the antibody.

A chimeric antibody containing variable regions from the murine CLB-8 antibody has been demonstrated in accordance with the present invention to be as effective as murine monoclonal antibody CLB-8 in binding to IL-6.

The human constant region of the chimeric antibody of the invention can be of any class (IgG, IgA, IgM, IgE, IgD, etc.) or isotype and can comprise a kappa or lambda light chain. In one embodiment, the human constant region comprises an IgG heavy chain or defined fragment, for example, at least one of isotypes, IgG1, IgG2, IgG3 or IgG4. In another embodiment, the anti-human IL-6 human antibody comprises an IgG1 heavy chain and a IgG1 K light chain. The isolated anti-IL-6 antibodies of the present invention comprise antibody amino acid sequences disclosed herein encoded by any suitable polynucleotide as well as. Preferably, the antibody or antigen-binding fragment binds human IL-6 and, thereby partially or substantially neutralizes at least one biological activity of the protein. The cCLB-8 antibody, or specified portion or variant thereof, partially or preferably substantially neutralizes at least one biological activity of at least one IL-6 protein or fragment and thereby inhibit activities mediated through the binding of IL-6 to the IL-6 receptor or through other IL-6-dependent or mediated mechanisms. As used herein, the term “neutralizing antibody” refers to an antibody that can inhibit an IL-6-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-6 antibody to inhibit an IL-6-dependent activity is preferably assessed by at least one suitable IL-6 protein or receptor assay, as described herein and/or as known in the art.

At least one antibody of the invention binds at least one specified epitope specific to at least one IL-6 protein, subunit, fragment, portion or any combination thereof to which the CLB-8 antibody binds. The at least one epitope can comprise at least one antibody binding region that comprises at least one portion of the protein, which epitope is preferably comprised of at least one extracellular, soluble, hydrophillic, external or cytoplasmic portion of the protein. Generally, the human antibody or antigen-binding fragment of the present invention will comprise an antigen-binding region that comprises at least one human complementarity determining region (CDR1, CDR2 and CDR3) of SEQ ID NOS. 141, 142 and 143 or variant of at least one heavy chain variable region and at least one human complementarity determining region (CDR4, CDR5 and CDR6) (SEQ ID NO. 144, 145 and 146) or variant of at least one light chain variable region. As a non-limiting example, the antibody or antigen-binding portion or variant can comprise at least one of the heavy chain CDR3 having the amino acid sequence of SEQ ID NO:143, and/or a light chain CDR3 having the amino acid sequence of SEQ ID NO:146. In a particular embodiment, the antibody or antigen-binding fragment can have an antigen-binding region that comprises at least a portion of at least one heavy chain CDR (i.e., CDR1, CDR2 and/or CDR3) having the amino acid sequence of the corresponding CDRs 1, 2 and/or 3 (e.g., SEQ ID NOS:141, 142, and/or 143). In another particular embodiment, the antibody or antigen-binding portion or variant can have an antigen-binding region that comprises at least a portion of at least one light chain CDR (i.e., CDR4, CDR5 and/or CDR6) having the amino acid sequence of the corresponding CDRs 4, 5 and/or 6 (e.g., SEQ ID NOS: 144, 145, and/or 146). In a preferred embodiment the three heavy chain CDRs and the three light chain CDRs of the antibody or antigen-binding fragment have the amino acid sequence of the corresponding CDR of at least one of mAb cCLB8, Chimeric anti-IL-6 Mab, as described herein. Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method and using any of the possible redundant codons that will result in expression of a polypeptide of the invention.

Antibodies that bind to human IL-6 and that comprise the defined heavy or light chain variable region or CDR regions can be prepared using suitable methods, such as phage display (Katsube, Y., et al., Int J Mol. Med, 1(5):863-868 (1998)) or methods that employ transgenic animals, as known in the art and/or as described herein. For example, the antibody, specified portion or variant can be expressed using the encoding nucleic acid or portion thereof in a suitable host cell.

As stated, the invention also relates to antibodies, antigen-binding fragments, immunoglobulin chains and CDRs comprising amino acids in a sequence that is substantially the same as an amino acid sequence described herein. Such anti-IL-6 antibodies can include one or more amino acid substitutions, deletions or additions, either from natural mutations or human manipulation, as specified herein. Preferably, such antibodies or antigen-binding fragments and antibodies comprising such chains or CDRs can bind human IL-6 with high affinity (e.g., KD less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Of course, the number of amino acid substitutions a skilled artisan would make depends on many factors, including those described above. Generally speaking, the number of amino acid substitutions, insertions or deletions for any given anti-IL-6 antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-IL-6 antibody of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one IL-6 neutralizing activity. Sites that are critical for antibody binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

Anti-IL-6 antibodies of the present invention can include, but are not limited to, at least one portion, sequence or combination selected from 5 to all of the contiguous amino acids of at least one of SEQ ID NOS:141, 142, 143, 144, 145, 146.

An anti-IL-6 antibody can further optionally comprise a polypeptide of at least one of 70-100% of the contiguous amino acids of at least one of SEQ ID NOS: 139 and 140.

In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of SEQ ID NOS:9, 10. For example, the amino acid sequence of a light chain variable region can be compared with the sequence of SEQ ID NO:10, or the amino acid sequence of a heavy chain CDR3 can be compared with SEQ ID NO: 139, 140. Preferably, 70-100% amino acid identity (i.e., 90, 91, 92, 93, 94, 95, 96, 97, 98, 99, 100 or any range or value therein) is determined using a suitable computer algorithm, as known in the art.

Exemplary heavy chain and light chain variable regions sequences are provided in SEQ ID NOS: 139, 140. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL-6 antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and preferably at least 50%, 60%, or 70%, and most preferably at least 80%, 90%, or 95%-1000% of that of the native (non-synthetic), endogenous or related and known antibody. Methods of assaying and quantifying measures of enzymatic activity and substrate specificity, are well known to those of skill in the art.

In another aspect, the invention relates to antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrrolidone, and the fatty acid or fatty acid ester group can comprise from about eight to about forty carbon atoms.

The modified antibodies and antigen-binding fragments of the invention can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-49-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C1-C12 group wherein one or more carbon atoms can be replaced by a heteroatom such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2-O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

The antibodies of the invention can bind human IL-6 with a wide range of affinities (KD). In a preferred embodiment, at least one human mAb of the present invention can optionally bind human IL-6 with high affinity. For example, a mAb can bind human IL-6 with a KD equal to or less than about 10-7 M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., KD, Ka, Kd) are preferably made with standardized solutions of antibody and antigen, and a standardized buffer, such as the buffer described herein.

Anti-IL-6 cCLB-8 antibodies useful in the methods and compositions of the present invention are characterized by high affinity binding to IL-6 and optionally and preferably having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference).

When cCLB8 is compared to other IL-6-specific antibodies CLB.IL-6/14 and CLB.IL-6/16, one can see the distinct characteristics of antibody affinity and epitope specificity. cCLB8, an antibody that binds IL-6 and normally blocks the interaction between IL-6 and its receptor, can inhibit nearly 100% of IL-6 function as illustrated in both the IL-6 dependent 7TD1 cell proliferation bioassay and the IL-6 binding to IL-6 receptor Luminex based assay. In contrast, CLB.IL-6/16, an antibody that binds IL-6, but neutralizes by sterically hindering the interaction between the IL-6/IL-6R complex and the gp130 signaling component, can inhibit only 62% of the bound biotin-IL-6. Finally, an antibody that binds IL-6 but does not interfere with its biological activity, as in CLB.IL-6/14, displays no inhibition of biotin-IL-6 binding the solid phase sIL-6R/gp80.

Bispecific, heterospecific, heteroconjugate or similar antibodies can also be used that are monoclonal, humanized, antibodies that have binding specificities for at least two different antigens. In the present case, one of the binding specificities is for at least one IL-6 protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

### Human of Human Engineered Monoclonal Antibodies

In one aspect of the invention there is provided a method for the treatment or prophylaxis of an IL-6-mediated disorder such as depression and/or fatigue, comprising administering to a patient in need thereof a therapeutically effective amount of an IL-6 antigen binding protein or fragment thereof.

In one such aspect of the invention as herein described the antigen binding protein or fragment thereof specifically binds to IL-6 and inhibits the binding of IL-6 to the IL-6 receptor (IL-6R).

In one aspect of the invention there is provided a method for the treatment or prophylaxis of an IL-6-mediated disorder such as depression and/or fatigue comprising administering to a patient in need thereof a therapeutically effective amount of an IL-6 antigen binding protein or fragment thereof wherein the antigen binding protein or fragment thereof comprises one or more of the following CDR's:

### i) CDRH1 as set out in SEQ ID NO. 135; or

### ii) CDRH2 as set out in SEQ ID NO. 136; or

iii) CDRH3 as set out in SEQ ID NO. 137; or

### iv) CDRL1 as set out in SEQ ID NO. 132; or

### v) CDRL2 as set out in SEQ ID NO. 133; or

vi) CDRL3 as set out in SEQ ID NO. 134; and wherein:
X₁ is A or G, X₂ is S or R, X₃ is H, I, S, or Y, X₄ is S or Y, X₅ is S or F, X₆ is F, L, M, or T, X₇ is N or E, X₈ is A or T, X₉ is M, C, S or Q, X₁₀ is Q or C, X₁₁ is T or Q, X₁₂ is F, S, or T, X₁₃ is S or P, X₁₄ is L or M, X₁₅ is A or I, X₁₆ is S or P, X₁₇ is Y or W, X₁₈ is T, E, or Y, X₁₉ is Y or F, X₂₀ is P, S, D, or Y, X₂₁ is V or D, X₂₂ is T or A, X₂₃ is G or P, X₂₄ is S, Y, T, or N, and X₂₅ is Y, T, F, or I.

In a further aspect the antigen binding protein comprises

### i) CDRH1 as set out in SEQ ID NO. 135; or

### ii) CDRH2 as set out in SEQ ID NO. 136; or

iii) CDRH3 as set out in SEQ ID NO. 137; or

### iv) CDRL1 as set out in SEQ ID NO. 132; or

### v) CDRL2 as set out in SEQ ID NO. 133; or

vi) CDRL3 as set out in SEQ ID NO. 134; and wherein:
X₁ is A or G, X₂ is S or R, X₃ is H, I, S, or Y, X₄ is S or Y, X₅ is S or F, X₆ is F, L, M, or T, X₇ is N or E, X₈ is A or T, X₉ is M, C, S or Q, X₁₀ is Q or C, X₁₁ is T or Q, X₁₂ is F, S, or T, X₁₃ is S or P, X₁₄ is L or M, X₁₅ is A or I, X₁₆ is S or P, X₁₇ is Y or W, X₁₈ is T, E, or Y, X₁₉ is Y or F, X₂₀ is P, S, D, or Y, X₂₁ is V or D, X₂₂ is T or A, X₂₃ is G or P, X₂₄ is S, Y, T, or N, and X₂₅ is Y, T, F, or I.

In yet a further aspect of the invention as herein described the antigen binding protein or fragment thereof comprises the following CDR's:

a CDRH1 of SEQ ID NO:135 comprising the sequence G-F-X₁₁-X₁₂-S-X₁₃-F-A-X₁₄-S, wherein X₁₁ is T or Q, X₁₂ is F, S, or T, X₁₃ is S or P, and X₁₄ is L or M; and

a CDRH2 of SEQ ID NO:136 comprising the sequence K-X₁₅-S-X₁₆-G-G-S-X₁₇-X₁₈-Y-X₁₉-X₂₀-D-TX₂₁-X₂₂-X₂₃, wherein X₁₅ is A or I, X₁₆ is S or P, X₁₇ is Y or W, X₁₈ is T, E, or Y, X₁₉ is Y or F, X₂₀ is P, S, D, or F, X₂₁ is V or D, X₂₂ is T or A, and X₂₃ is G or P; and a CDRH3 amino acid sequence of SEQ ID NO:137 comprising the sequence Q-L-W-G-X₂₄-Y-A-L-D-X₂₅, wherein X₂₄ is S, Y, T, or N, and X₂₅ is Y, T, F, or I; and

CDRL1 of SEQ ID NO:132 comprising the sequence S-X₁-X₂-X₃-X₄-V-X₅-Y-M-Y, wherein X₁ is A or G, X₂ is S or R, X₃ is H, I, S, or Y, X₄ is S or Y, and X₅ is S or F; and

a CDRL2 of SEQ ID NO:133 comprising the sequence D-X₆-S-X₇-L-X₈-S, wherein X₆ is F, L, M, or T, X₇ is N or E, and X₈ is A or T; and

a CDRL3 of SEQ ID NO:134 comprising the sequence X₉-X₁₀-W-S-G-Y-P-Y-T, wherein X₉ is M, C, or S, and X₁₀ is Q or C.

In one aspect the antigen binding protein or fragment thereof has one or more of the following CDR sequences:

CDRH1 according to SEQ ID NO:39

CDRH2 according to SEQ ID NO:59

CDRH3 according to SEQ ID NO:89

CDRL1 according to SEQ ID NO:3

CDRL2 according to SEQ ID NO:21 or

CDRL3 according to SEQ ID NO:29

In a further aspect the IL-6 antigen binding protein or fragment thereof comprises CDRH1 according to SEQ ID NO:39 and CDRH2 according to SEQ ID NO:59 and CDRH3 according to SEQ ID NO:89.

In yet a further aspect the IL-6 antigen binding protein or fragment thereof comprises CDRH1 according to SEQ ID NO:39 and CDRH2 according to SEQ ID NO:59 and CDRH3 according to SEQ ID NO:89 and CDRL1 according to SEQ ID NO:3 and CDRL2 according to SEQ ID NO:21 and CDRL3 according to SEQ ID NO:29.

In one aspect of the invention as herein described the IL-6 antibodies of the invention as herein described have the sequences shown in Tables 1-9 below. For example, an anti-IL-6 antibody of the invention has one of the light chain CDR sequences shown in Table 1 (i.e., CDRL1, CDRL2, and CDRL3) and one of the heavy chain CDR sequences shown in Table 2 (i.e., CDRH1, CDRH2, and CDRH3). More specifically, an anti-IL-6 antibody (for example molecule AME-19a) has the CDRL1 of SEQ ID NO:3, CDRL2 of SEQ ID NO:21, CDRL3 of SEQ ID NO:29, CDRH1 of SEQ ID NO:39, CDRH2 of SEQ ID NO:59, CDRH3 of SEQ ID NO:89.

In a preferred aspect, the three heavy chain CDRs and the three light chain CDRs of the antibody or antigen-binding fragment have the amino acid sequence of the corresponding CDR of at least one of mAb AME-A9, AME-1b, AME-18a, AME-22a, AME-20b, AME-23a, and AME-19a, as described herein. Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The antigen binding proteins of the invention may comprise heavy chain variable regions and light chain variable regions of the invention which may be formatted into the structure of a natural antibody or functional fragment or equivalent thereof. An antigen binding protein of the invention may therefore comprise the VH regions of the invention formatted into a full length antibody, a (Fab′)2 fragment, a Fab fragment, or equivalent thereof (such as scFV, bitri- or tetra-bodies, Tandabs etc.), when paired with an appropriate light chain.

In one such aspect of the invention as herein described the antigen binding protein is selected from the group consisting of a dAb, Fab, Fab′, F(ab′)2, Fv, diabody, triabody, tetrabody, miniantibody, and a minibody.

The antigen binding protein or human engineered IL-6 antibody of the present invention may comprise a human germline light chain framework. In particular aspects, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain aspects, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6. See PCT WO 2005/005604 for a description of the different germline sequences.

In other aspects, the antigen binding protein or human engineered IL-6 antibody of the present invention may comprise a human germline heavy chain framework. In particular aspects, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81. See PCT WO2005/005604 for a description of the different germline sequences.

In particular aspects, the light chain variable region and/or heavy chain variable region comprises a framework region or at least a portion of a framework region (e.g., containing 2 or 3 sub regions, such as FR2 and FR3). In certain aspects, at least FRL1, FRL2, FRL3, or FRL4 is fully human. In other aspects, at least FRH1, FRH2, FRH3, or FRH4 is fully human. In some aspects, at least FRL1, FRL2, FRL3, or FRL4 is a germline sequence (e.g., human germline) or comprises human consensus sequences for the particular framework (readily available at the sources of known human Ig sequences described above). In other aspects, at least FRH1, FRH2, FRH3, or FRH4 is a germline sequence (e.g., human germline) or comprises human consensus sequences for the particular framework. In preferred aspects, the framework region is a human framework region (e.g., the human framework regions shown below in Table 9). In some aspects, the framework region comprises SEQ ID NOS: 105, 106, 107, 108, 109, 110, 111, 112, or combinations thereof

In one aspect of the invention there is provided an antigen binding protein comprising an isolated heavy chain variable domain of SEQ ID NO: 99.

In another aspect of the invention there is provided an antigen binding protein comprising an isolated light chain variable domain SEQ ID NO: 97.

In a further aspect of the invention there is provided an antigen binding protein comprising an isolated heavy chain variable domain of SEQ ID NO: 99 and a an isolated light chain variable domain of SEQ ID NO.97. For example in one such aspect the IL-6 antigen binding protein or fragment thereof is CNTO136 for example the IL-6 antigen binding protein or fragment thereof is Sirukumab.

The anti-IL-6 antibody can comprise at least one of a heavy or light chain variable region having a defined amino acid sequence. For example, in a preferred aspect, the anti-IL-6 antibody comprises at least one of at least one heavy chain variable region, optionally having an amino acid sequence selected from the group consisting of SEQ ID NOS:95, 99, 103, 118, 122, 126, and 130, and/or at least one light chain variable region, optionally having an amino acid sequence selected from the group consisting of SEQ ID NOS:93, 97, 101, 116, 120, 124, and 128. Antibodies that bind to human IL-6 and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL-6 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

In a further aspect the antigen binding protein may comprise any one of the variable heavy chains as described in Table 6 herein in combination with any one of the light chains as described in Table 6 herein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype.

Alternatively or additionally, it may be useful to combine amino acid modifications with one or more further amino acid modifications that alter C1q binding and/or the complement dependent cytotoxicity (CDC) function of the Fc region of an IL-6 binding molecule. The starting polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human engineered IL-6 antibody of the present invention with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of the human engineered IL-6 antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human engineered IL-6 antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of a human engineered IL-6 antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human engineered IL-6 antibody of the present invention is expressed in cells that express beta (1,4)-N-acetylglucosaminyltransferase III (GnT III), such that GnT III adds GlcNAc to the human engineered IL-6 antibody. Methods for producing antibodies in such a fashion are provided in WO/9954342, WO/03011878, patent publication 20030003097A1, and Umana et al., Nature Biotechnology, 17:176-180, February 1999.

A human anti-IL-6 antibody can be optionally generated by immunization of a transgenic animal (e.g., mouse, rat, hamster, non-human primate, and the like) capable of producing a repertoire of human antibodies, as described herein and/or as known in the art. Cells that produce a human anti-IL-6 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, 5427908, 5580717, assigned to Affymax; 5885793, assigned to Cambridge Antibody Technologies; 5750373, assigned to Genentech, 5618920, 5595898, 5576195, 5698435, 5693493, 5698417, assigned to Xoma, Colligan, supra; Ausubel, supra; or Sambrook, supra.

Antibodies of the present invention can also be prepared using at least one anti-IL-6 antibody encoding nucleic acid to provide transgenic animals or mammals, such as goats, cows, horses, sheep, rabbits and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies of the present invention can additionally be prepared using at least one anti-IL-6 antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein.

The antibodies of the invention can bind human IL-6 with a wide range of affinities (KD). In a preferred embodiment, at least one human mAb of the present invention can optionally bind human IL-6 with high affinity. For example, a human or human engineered mAb can bind human IL-6 with a KD equal to or less than about 10-7 M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., KD, Kon, Koff) are preferably made with standardized solutions of antibody and antigen, and a standardized buffer, such as the buffer described herein.

Preferred anti-IL-6 antibodies of the invention have the sequences shown in Tables 1-9 below. For example, an anti-IL-6 antibody of the invention has one of the light chain CDR sequences shown in Table 1 (i.e., CDRL1, CDRL2, and CDRL3) and one of the heavy chain CDR sequences shown in Table 2 (i.e., CDRH1, CDRH2, and CDRH3). More specifically, an anti-IL-6 antibody (molecule AME-A9) has the CDRL1 of SEQ ID NO:15, CDRL2 of SEQ ID NO:27, CDRL3 of SEQ ID NO:35, CDRH1 of SEQ ID NO:47, CDRH2 of SEQ ID NO:61, CDRH3 of SEQ ID NO:91.

TABLE 1
Light Chain CDRs

| | CDR | | |
|---|---|---|---|
| SEQ ID NO | Name* | Clone | Sequence |
| SEQ ID NO: 1 | CDRL1 | 33 | SASHSVSYMY |
| SEQ ID NO: 2 | CDRL1 | 33 | AGTGCCAGCCATAGTGTAAGTTACATGTAC |
| SEQ ID NO: 3 | CDRL1 | 34 | SASISVSYMY |
| SEQ ID NO: 4 | CDRL1 | 34 | AGTGCCAGCATTAGTGTAAGTTACATGTAC |
| SEQ ID NO: 5 | CDRL1 | 35 | SARSSVSYMY |
| SEQ ID NO: 6 | CDRL1 | 35 | AGTGCCCGGTCAAGTGTAAGTTACATGTAC |
| SEQ ID NO: 7 | CDRL1 | 36 | SASYSVSYMY |
| SEQ ID NO: 8 | CDRL1 | 36 | AGTGCCAGCTATAGTGTAAGTTACATGTAC |
| SEQ ID NO: 9 | CDRL1 | 37 | SASSSVFYMY |
| SEQ ID NO: 10 | CDRL1 | 37 | AGTGCCAGCTCAAGTGTATTTTACATGTAC |
| SEQ ID NO: 11 | CDRL1 | 39 | SGSSYVSYMY |
| SEQ ID NO: 12 | CDRL1 | 39 | AGTGGCAGCTCATATGTAAGTTACATGTAC |
| SEQ ID NO: 13 | CDRL1 | 40 | SALSSVSYMY |
| SEQ ID NO: 14 | CDRL1 | 40 | AGTGCCCTGTCAAGTGTAAGTTACATGTAC |
| SEQ ID NO: 15 | CDRL1 | A9 | SASSSVSYMY |
| SEQ ID NO: 16 | CDRL1 | A9 | AGTGCCAGCTCAAGTGTAAGTTACATGTAC |
| SEQ ID NO: 17 | CDRL2 | 41 | DFSNLAS |
| SEQ ID NO: 18 | CDRL2 | 41 | GACTTTTCCAACCTGGCTTCT |
| SEQ ID NO: 19 | CDRL2 | 43 | DLSNLAS |
| SEQ ID NO: 20 | CDRL2 | 43 | GACCTGTCCAACCTGGCTTCT |
| SEQ ID NO: 21 | CDRL2 | 44 | DMSNLAS |
| SEQ ID NO: 22 | CDRL2 | 44 | GACATGTCCAACCTGGCTTCT |
| SEQ ID NO: 23 | CDRL2 | 46 | DTSNLTS |
| SEQ ID NO: 24 | CDRL2 | 46 | GACACATCCAACCTGACGTCT |
| SEQ ID NO: 25 | CDRL2 | 48 | DTSELAS |
| SEQ ID NO: 26 | CDRL2 | 48 | GACACATCCGAGCTGGCTTCT |
| SEQ ID NO: 27 | CDRL2 | A9 | DTSNLAS |
| SEQ ID NO: 28 | CDRL2 | A9 | GACACATCCAACCTGGCTTCT |
| SEQ ID NO: 29 | CDRL3 | 49 | MQWSGYPYT |
| SEQ ID NO: 30 | CDRL3 | 49 | ATGCAGTGGAGTGGTTACCCATACACG |
| SEQ ID NO: 31 | CDRL3 | 50 | CQWSGYPYT |
| SEQ ID NO: 32 | CDRL3 | 50 | TGTCAGTGGAGTGGTTACCCATACACG |
| SEQ ID NO: 33 | CDRL3 | 52 | SCWSGYPYT |
| SEQ ID NO: 34 | CDRL3 | 52 | TCTGTGTGGAGTGGTTACCCATACACG |
| SEQ ID NO: 35 | CDRL3 | A9 | SQWSGYPYT |
| SEQ ID NO: 36 | CDRL3 | A9 | TCTCAGTGGAGTGGTTACCCATACACG |
| SEQ ID NO: 138 | CDRL3 | Alt. | QQWSGYPYT |

*CDRs were as defined by Kabat with the exception of CDRH1 which is the sum of Kabat and Chothia definitions.

TABLE 2
Heavy Chain CDRs

| | CDR | | |
|---|---|---|---|
| SEQ ID NO | Name* | Clone | Sequence |
| SEQ ID NO: 37 | CDRH1 | 4 | GFTFSSFALS |
| SEQ ID NO: 38 | CDRH1 | 4 | GGATTCACCTTTAGTAGCTTTGCCCTTTCT |
| SEQ ID NO: 39 | CDRH1 | 5 | GFTFSPFAMS |
| SEQ ID NO: 40 | CDRH1 | 5 | GGATTCACCTTTAGTCCTTTTGCCATGTCT |
| SEQ ID NO: 41 | CDRH1 | 6 | GFQFSSFAMS |
| SEQ ID NO: 42 | CDRH1 | 6 | GGATTCCAGTTTAGTAGCTTTGCCATGTCT |
| SEQ ID NO: 43 | CDRH1 | 8 | GFTTSSFAMS |
| SEQ ID NO: 44 | CDRH1 | 8 | GGATTCACCACTAGTAGCTTTGCCATGTCT |
| SEQ ID NO: 45 | CDRH1 | Q + P | GFQFSPFAMS |
| SEQ ID NO: 46 | CDRH1 | Q + P | GGATTCCAGTTTAGTCCTTTTGCCATGTCT |
| SEQ ID NO: 47 | CDRH1 | A9 | GFTFSSFAMS |
| SEQ ID NO: 48 | CDRH1 | A9 | GGATTCACCTTTAGTAGCTTTGCCATGTCT |
| SEQ ID NO: 49 | CDRH2 | 10 | KASSGGSYTYYPDTVTG |
| SEQ ID NO: 50 | CDRH2 | 10 | AAAGCGAGTAGTGGTGGGAGTTACACCTACTATCCTGA |
| | | | CACTGTGACGGGC |
| SEQ ID NO: 51 | CDRH2 | 11 | KISSGGSYEYYPDTVTG |
| SEQ ID NO: 52 | CDRH2 | 11 | AAAATTAGTAGTGGTGGGAGTTACGAGTACTATCCTGA |
| | | | CACTGTGACGGGC |
| SEQ ID NO: 53 | CDRH2 | 12 | KISSGGSYYYYPDTVTG |
| SEQ ID NO: 54 | CDRH2 | 12 | AAAATTAGTAGTGGTGGGAGTTACTATTACTATCCTGA |
| | | | CACTGTGACGGGC |
| SEQ ID NO: 55 | CDRH2 | 14 | KISSGGSWTYYPDTVTG |
| SEQ ID NO: 56 | CDRH2 | 14 | AAAATTAGTAGTGGTGGGAGTTGGACCTACTATCCTGA |
| | | | CACTGTGACGGGC |
| SEQ ID NO: 57 | CDRH2 | 16 | KISPGGSYTYYPDTVTG |
| SEQ ID NO: 58 | CDRH2 | 16 | AAAATTAGTCCGGGTGGGAGTTACACCTACTATCCTGA |
| | | | CACTGTGACGGGC |
| SEQ ID NO: 59 | CDRH2 | P + W + | KISPGGSWTYYSDTVTG |
| | | S (18a, | |
| | | 19a) | |
| SEQ ID NO: 60 | CDRH2 | P + W + | AAAATTAGTCCGGGTGGGAGTTGGACCTACTATTCTGA |
| | | S (18a, | CACTGTGACGGGC |
| | | 19a) | |
| SEQ ID NO: 61 | CDRH2 | A9 | KISSGGSYTYYPDTVTG |
| SEQ ID NO: 62 | CDRH2 | A9 | AAAATTAGTAGTGGTGGGAGTTACACCTACTATCCTGA |
| | | | CACTGTGACGGGC |
| SEQ ID NO: 113 | CDRH2 | Alt. | EISSGGSYTYYPDTVTG |
| SEQ ID NO: 63 | CDRH2 | 17 | KISSGGSYTYFPDTVTG |
| SEQ ID NO: 64 | CDRH2 | 17 | AAAATTAGTAGTGGTGGGAGTTACACCTACTTTCCTGA |
| | | | CACTGTGACGGGC |
| SEQ ID NO: 65 | CDRH2 | 19 | KISSGGSYTYYPDTVAG |
| SEQ ID NO: 66 | CDRH2 | 19 | AAAATTAGTAGTGGTGGGAGTTACACCTACTATCCTGA |
| | | | CACTGTGGCTGGC |
| SEQ ID NO: 67 | CDRH2 | 20 | KISSGGSYTYYDDTVTG |
| SEQ ID NO: 68 | CDRH2 | 20 | AAAATTAGTAGTGGTGGGAGTTACACCTACTATGATGA |
| | | | CACTGTGACGGGC |
| SEQ ID NO: 69 | CDRH2 | 21 | KISSGGSYTYYSDTVTG |
| SEQ ID NO: 70 | CDRH2 | 21 | AAAATTAGTAGTGGTGGGAGTTACACCTACTATTCTGA |
| | | | CACTGTGACGGGC |
| SEQ ID NO: 71 | CDRH2 | 22 | KISSGGSYTYYPDTVTP |
| SEQ ID NO: 72 | CDRH2 | 22 | AAAATTAGTAGTGGTGGGAGTTACACCTACTATCCTGA |
| | | | CACTGTGACGCCG |
| SEQ ID NO: 73 | CDRH2 | 23 | KISSGGSYTYYPDTDTG |
| SEQ ID NO: 74 | CDRH2 | 23 | AAAATTAGTAGTGGTGGGAGTTACACCTACTATCCTGA |
| | | | CACTGATACGGGC |
| SEQ ID NO: 75 | CDRH2 | P + S | KISPGGSYTYYSDTVTG |
| | | (20b, | |
| | | 23a) | |
| SEQ ID NO: 76 | CDRH2 | P + S | AAAATTAGTCCGGGTGGGAGTTACACCTACTATTCTGA |
| | | (20b, | CACTGTGACGGGC |
| | | 23a) | |
| SEQ ID NO: 77 | CDRH2 | P + W + | KISPGGSWTYYDDTVTG |
| | | D (22a) | |
| SEQ ID NO: 78 | CDRH2 | P + W + | AAAATTAGTCCGGGTGGGAGTTGGACCTACTATGATGA |
| | | D (22a) | CACTGTGACGGGC |
| SEQ ID NO: 79 | CDRH3 | 25 | QLWGSYALDY |
| SEQ ID NO: 80 | CDRH3 | 25 | CAGTTATGGGGGTCGTATGCTCTTGACTAC |
| SEQ ID NO: 81 | CDRH3 | 26 | QLWGYYALDT |
| SEQ ID NO: 82 | CDRH3 | 26 | CAGTTATGGGGGTACTATGCTCTTGACACG |
| SEQ ID NO: 83 | CDRH3 | 29 | QLWGTYALDY |
| SEQ ID NO: 84 | CDRH3 | 29 | CAGTTATGGGGGACTTATGCTCTTGACTAC |
| SEQ ID NO: 85 | CDRH3 | 30 | QLWGNYALDY |
| SEQ ID NO: 86 | CDRH3 | 30 | CAGTTATGGGGGAATTATGCTCTTGACTAC |
| SEQ ID NO: 87 | CDRH3 | 31 | QLWGYYALDF |
| SEQ ID NO: 88 | CDRH3 | 31 | CAGTTATGGGGGTACTATGCTCTTGACTTT |
| SEQ ID NO: 89 | CDRH3 | 32 | QLWGYYALDI |
| SEQ ID NO: 90 | CDRH3 | 32 | CAGTTATGGGGGTACTATGCTCTTGACATT |
| SEQ ID NO: 91 | CDRH3 | A9 | QLWGYYALDY |
| SEQ ID NO: 92 | CDRH3 | A9 | CAGTTATGGGGGTACTATGCTCTTGACTAC |
| SEQ ID NO: 114 | CDRH3 | Alt. | GLWGYYALDY |

*CDRs were as defined by Kabat with the exception of CDRH1 which is the sum of Kabat and Chothia definitions.

TABLE 3
Mutations from Individual CDR libraries

| Clone | |
|---|---|
| | CDRH1 |
| 4 | M34L |
| 5 | S31P |
| 6 | T28Q |
| 8 | F29T |
| | CDRH2 |
| 10 | I51A |
| 11 | T57E |
| 12 | T57Y |
| 14 | Y56W |
| 16 | S52aP |
| 17 | Y59F |
| 19 | T64A |
| 20 | P60D |
| 21 | P60S |
| 22 | G65P |
| 23 | V63D |
| | CDRH3 |
| 25 | Y99S |
| 26 | Y102T |
| 27 | Y99S |
| 29 | Y99T |
| 30 | Y99N |
| 31 | Y102F |
| 32 | Y102I |
| | CDRL1 |
| 33 | S27H |
| 34 | S27I |
| 35 | S26R |
| 36 | S27Y |
| 37 | S30F |
| 38 | S27I |
| 39 | A25G, S28Y |
| 40 | S26L |
| | CDRL2 |
| 41 | T51F |
| 43 | T51L |
| 44 | T51M |
| 46 | A55T |
| 47 | T51L |
| 48 | N53E |
| | CDRL3 |
| 49 | Q89M |
| 50 | Q89C |
| 52 | Q90C |

TABLE 4
Mutations Included in the Combinatorial Library

| CDRH1 | CDRH2 | CDRH3 | CDRL1 | CDRL2 | CDRL3 |
|---|---|---|---|---|---|
| T28Q | S52aP | Y102F | S27I | T51F | Q89M |
| S31P | Y56W | Y102I | S27Y | T51M | |
| | P60S | | | | |
| | V63D | | | | |

TABLE 5A
Positive Library Clones

| | Light | | | Heavy | | | | | | | | |
|---|---|---|---|---|---|---|---|---|---|---|---|---|
| CDR--> | L1 | L2 | L3 | H1 | | H2 | | | | | H3 | |
| WT--> | S | T | Q | T | S | E | S | Y | P | V | G | Y |
| CNTO328 | | | | | | | | | | | | |
| Clone | 27 | 51 | 89 | 28 | 31 | 50 | | 56 | 60 | 63 | 95 | 102 |
| | | | | | | | 52a | | | | | |
| AME-A9 | | | S | | | K | | | | | Q | |
| AME-16 | | | S | | | K | P | | | | Q | |
| AME-18a | | F | M | Q | P | K | P | W | S | | Q | I |
| AME-19a | I | M | M | | P | K | P | W | S | | Q | I |
| AME-20b | I | M | M | Q | | K | P | | S | | Q | I |
| AME-22a | Y | F | M | Q | P | K | P | W | | D | Q | F |
| AME-23a | Y | M | M | Q | | K | P | | S | | Q | F |

TABLE 5B
Human Engineered Anti-IL-6 Antibody Clones and Corresponding CDRs

| | CDR | | | | | |
|---|---|---|---|---|---|---|
| | L1 | L2 | L3 | H1 | H2 | H3 |
| AME-A9 | SEQ ID: 15 | SEQ ID: 27 | SEQ ID: 35 | SEQ ID: 47 | SEQ ID: 61 | SEQ ID: 91 |
| AME-16 | SEQ ID: 15 | SEQ ID: 27 | SEQ ID: 35 | SEQ ID: 47 | SEQ ID: 57 | SEQ ID: 91 |
| AME-18a | SEQ ID: 15 | SEQ ID: 17 | SEQ ID: 29 | SEQ ID: 45 | SEQ ID: 59 | SEQ ID: 89 |
| AME-19a | SEQ ID: 3 | SEQ ID: 21 | SEQ ID: 29 | SEQ ID: 39 | SEQ ID: 59 | SEQ ID: 89 |
| AME-20b | SEQ ID: 3 | SEQ ID: 21 | SEQ ID: 29 | SEQ ID: 41 | SEQ ID: 75 | SEQ ID: 89 |
| AME-22a | SEQ ID: 7 | SEQ ID: 17 | SEQ ID: 29 | SEQ ID: 45 | SEQ ID: 77 | SEQ ID: 87 |
| AME-23a | SEQ ID: 7 | SEQ ID: 21 | SEQ ID: 29 | SEQ ID: 41 | SEQ ID: 75 | SEQ ID: 87 |

TABLE 6
Variable region sequences of clones

| | | Heavy | |
|---|---|---|---|
| | | (H) or | |
| | | Light (L) | |
| SEQ ID | | Chain V | |
| NO | Clone | Region | Sequence |
| 93 | A9 | L Chain AA | EIVLTQSPATLSLSPGERATLSCSASSSVS |
| | | | YMYWYQQIKPGQAPRLLIYDTSNLASGIPAR |
| | | | FSGSGSGTDFTLTISSLEPEDFAVYYCSQW |
| | | | SGYPYTEGGGTKVE1K |
| 94 | | L Chain | GAAATTGTGTTGACACAGTCTCCAGCCACCCTGTCTTTGTCTCCAGGGGA |
| | | Nucleotide | AAGAGCCACCCTCTCCTGCAGTGCCAGCTCAAGTGTAAGTTACATGTACT |
| | | | GGTACCAACAGAAACCTGGCCAGGCTCCCAGGCTCCTCATCTATGACACA |
| | | | TCCAACCTGGCTTCTGGCATCCCAGCCAGGTTCAGTGGCAGTGGGTCTGG |
| | | | GACAGACTTCACTCTCACCATCAGCAGCCTAGAGCCTGAAGATTTTGCAG |
| | | | TTTATTACTGTTCTCAGTGGAGTGGTTACCCATACACGTTCGGCGGAGGG |
| | | | ACCAAGGTGGAGATCAAA |
| 95 | | H Chain | EVQLVESGGGLVQPGGSLRLSCAASGFTFS |
| | | AA | SFAMSWVRQAPGEGLEWVAIKISSGGSYTYY |
| | | | PDTVTGRFTISRDNAKNSLYLQMNSLRAED |
| | | | TAVYYCARQLWGYYALDYWGQGTTVTVSS |
| 96 | | H Chain | GAGGTGCAGCTGGTGGAGTCTGGGGGAGGCTTGGTCCAGCCTGGGGGGTC |
| | | Nucleotide | CCTGAGACTCTCCTGTGCAGCCTCTGGATTCACCTTTAGTAGCTTTGCCA |
| | | | TGTCTTGGGTCCGCCAGGCTCCAGGGAAGGGGCTGGAGTGGGTGGCCAAA |
| | | | ATTAGTAGTGGTGGGAGTTACACCTACTATCCTGACACTGTGACGGGCCG |
| | | | ATTCACCATCTCCAGAGACAACGCCAAGAACTCACTGTATCTGCAAATGA |
| | | | ACAGCCTGAGAGCCGAGGACACGGCTGTGTATTACTGTGCGAGACAGTTA |
| | | | TGGGGGTACTATGCTCTTGACTACTGGGGCCAAGGGACCACGGTCACCGT |
| | | | CTCCTCA |
| 97 | 19A | L Chain AA | EIVLTQSPATLSLSPGERATLSCSASISVS |
| | | | YMYWYQQIKPGQAPRLLIYDMSNLASGIPAR |
| | | | FSGSGSGTDFTLTISSLEPEDFAVYYCMQW |
| | | | SGYPYTEGGGTKVE1K |
| 98 | | L Chain | GAAATTGTGTTGACACAGTCTCCAGCCACCCTGTCTTTGTCTCCAGGGGA |
| | | Nucleotide | AAGAGCCACCCTCTCCTGCAGTGCCAGCATTAGTGTAAGTTACATGTACT |
| | | | GGTACCAACAGAAACCTGGCCAGGCTCCCAGGCTCCTCATCTATGACATG |
| | | | TCCAACCTGGCTTCTGGCATCCCAGCCAGGTTCAGTGGCAGTGGGTCTGG |
| | | | GACAGACTTCACTCTCACCATCAGCAGCCTAGAGCCTGAAGATTTTGCAG |
| | | | TTTATTACTGTATGCAGTGGAGTGGTTACCCATACACGTTCGGCGGAGGG |
| | | | ACCAAGGTGGAGATCAAA |
| 99 | | H Chain | EVQLVESGGGLVQPGGSLRLSCAASGFTFS |
| | | AA | PFAMSWVRQAPGEGLEWVAIKISPGGSWTYY |
| | | | SDTVTGRFTISRDNAKNSLYLQMNSLRAED |
| | | | TAVYYCARQLWGYYALDIWGQGTTVTVSS |
| 100 | | H Chain | GAGGTGCAGCTGGTGGAGTCTGGGGGAGGCTTGGTCCAGCCTGGGGGGTC |
| | | Nucleotide | CCTGAGACTCTCCTGTGCAGCCTCTGGATTCACCTTTAGTCCTTTTGCCA |
| | | | TGTCTTGGGTCCGCCAGGCTCCAGGGAAGGGGCTGGAGTGGGTGGCCAAA |
| | | | ATTAGTCCGGGTGGGAGTTGGACCTACTATTCTGACACTGTGACGGGCCG |
| | | | ATTCACCATCTCCAGAGACAACGCCAAGAACTCACTGTATCTGCAAATGA |
| | | | ACAGCCTGAGAGCCGAGGACACGGCTGTGTATTACTGTGCGAGACAGTTA |
| | | | TGGGGGTACTATGCTCTTGACATTTGGGGCCAAGGGACCACGGTCACCGT |
| | | | CTCCTCA |
| 101 | 23A | L Chain AA | EIVLTQSPATLSLSPGERATLSCSASYSVS |
| | | | YMYWYQQIKPGQAPRLLIYDMSNLASGIPAR |
| | | | FSGSGSGTDFTLTISSLEPEDFAVYYCMQW |
| | | | SGYPYTEGGGTKVE1K |
| 102 | | L Chain | GAAATTGTGTTGACACAGTCTCCAGCCACCCTGTCTTTGTCTCCAGGGGA |
| | | Nucleotide | AAGAGCCACCCTCTCCTGCAGTGCCAGCTATAGTGTAAGTTACATGTACT |
| | | | GGTACCAACAGAAACCTGGCCAGGCTCCCAGGCTCCTCATCTATGACATG |
| | | | TCCAACCTGGCTTCTGGCATCCCAGCCAGGTTCAGTGGCAGTGGGTCTGG |
| | | | GACAGACTTCACTCTCACCATCAGCAGCCTAGAGCCTGAAGATTTTGCAG |
| | | | TTTATTACTGTATGCAGTGGAGTGGTTACCCATACACGTTCGGCGGAGGG |
| | | | ACCAAGGTGGAGATCAAA |
| 103 | | H Chain | EVQLVESGGGLVQPGGSLRLSCAASGFQFS |
| | | AA | SFAMSWVRQAPGEGLEWVAIKISPGGSYTYY |
| | | | SDTVTGRFTISRDNAKNSLYLQMNSLRAED |
| | | | TAVYYCARQLWGYYALDFWGQGTTVTVSS |
| 104 | | H Chain | GAGGTGCAGCTGGTGGAGTCTGGGGGAGGCTTGGTCCAGCCTGGGGGGTC |
| | | Nucleotide | CCTGAGACTCTCCTGTGCAGCCTCTGGATTCCAGTTTAGTAGCTTTGCCA |
| | | | TGTCTTGGGTCCGCCAGGCTCCAGGGAAGGGGCTGGAGTGGGTGGCCAAA |
| | | | ATTAGTCCGGGTGGGAGTTACACCTACTATTCTGACACTGTGACGGGCCG |
| | | | ATTCACCATCTCCAGAGACAACGCCAAGAACTCACTGTATCTGCAAATGA |
| | | | ACAGCCTGAGAGCCGAGGACACGGCTGTGTATTACTGTGCGAGACAGTTA |
| | | | TGGGGGTACTATGCTCTTGACTTTTGGGGCCAAGGGACCACGGTCACCGT |
| | | | CTCCTCA |
| 116 | AME-16 | L Chain AA | EIVLTQSPATLSLSPGERATLSCSASSSVS |
| | | | YMYWYQQIKPGQAPRLLIYDTSNLASGIPAR |
| | | | FSGSGSGTDFTLTISSLEPEDFAVYYCSQW |
| | | | SGYPYTEGGGTKVE1K |
| 117 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCTCAAGTGTAAGT |
| | | | TACATGTACTGGTACCAACAGAAACCTGGCCAGGCTCCCAGGCTCCTCAT |
| | | | CTATGACACATCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTTCTCAGTGGAGTGGTTACCCATACACGTT |
| | | | CGGCGGAGGGACCAAGGTGGAGATCAAA |
| 118 | | H Chain | EVQLVESGGGLVQPGGSLRLSCAASGFTFS |
| | | AA | SFAMSWVRQAPGEGLEWVAIKISPGGSYTYY |
| | | | PDTVTGRFTISRDNAKNSLYLQMNSLRAED |
| | | | TAVYYCARQLWGYYALDYWGQGTTVTVSS |
| 119 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCACCTTTAGTAGC |
| | | | TTTGCCATGTCTTGGGTCCGCCAGGCTCCAGGGAAGGGGCTGGAGTGGGT |
| | | | GGCCAAAATTAGTCCCGGTGGGAGTTACACCTACTATCCTGACACTGTGA |
| | | | CGGGCCGATTCACCATCTCCAGAGACAACGCCAAGAACTCACTGTATCTG |
| | | | CAAATGAACAGCCTGAGAGCCGAGGACACGGCTGTGTATTACTGTGCGAG |
| | | | ACAGTTATGGGGGTACTATGCTCTTGACTACTGGGGCCAAGGGACCACGG |
| | | | TCACCGTCTCCTCA |
| 120 | AME-18a | L Chain AA | EIVLTQSPATLSLSPGERATLSCSASSSVS |
| | | | YMYWYQQIKPGQAPRLLIYDFSNLASGIPAR |
| | | | FSGSGSGTDFTLTISSLEPEDFAVYYCMQW |
| | | | SGYPYTEGGGTKVE1K |
| 121 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCTCAAGTGTAAGT |
| | | | TACATGTACTGGTACCAACAGAAACCTGGCCAGGCTCCCAGGCTCCTCAT |
| | | | CTATGACTTCTCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTATGCAGTGGAGTGGTTACCCATACACGTT |
| | | | CGGCGGAGGGACCAAGGTGGAGATCAAA |
| 122 | | H Chain | EVQLVESGGGLVQPGGSLRLSCAASGFQFS |
| | | AA | PFAMSWVRQAPGEGLEWVAIKISPGGSWTYY |
| | | | SDTVTGRFTISRDNAKNSLYLQMNSLRAED |
| | | | TAVYYCARQLWGYYALDIWGQGTTVTVSS |
| 123 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCCAGTTTAGTCCC |
| | | | TTTGCCATGTCTTGGGTCCGCCAGGCTCCAGGGAAGGGGCTGGAGTGGGT |
| | | | GGCCAAAATTAGTCCCGGTGGGAGTTGGACCTACTATAGCGACACTGTGA |
| | | | CGGGCCGATTCACCATCTCCAGAGACAACGCCAAGAACTCACTGTATCTG |
| | | | CAAATGAACAGCCTGAGAGCCGAGGACACGGCTGTGTATTACTGTGCGAG |
| | | | ACAGTTATGGGGGTACTATGCTCTTGACATTTGGGGCCAAGGGACCACGG |
| | | | TCACCGTCTCCTCA |
| 124 | AME-20b | L Chain AA | EIVLTQSPATLSLSPGERATLSCSASISVS |
| | | | YMYWYQQIKPGQAPRLLIYDMSNLASGIPAR |
| | | | FSGSGSGTDFTLTISSLEPEDFAVYYCMQW |
| | | | SGYPYTEGGGTKVE1K |
| 125 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCATTAGTGTAAGT |
| | | | TACATGTACTGGTACCAACAGAAACCTGGCCAGGCTCCCAGGCTCCTCAT |
| | | | CTATGACATGTCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTATGCAGTGGAGTGGTTACCCATACACGTT |
| | | | CGGCGGAGGGACCAAGGTGGAGATCAAA |
| 126 | | H Chain | EVQLVESGGGLVQPGGSLRLSCAASGFQFS |
| | | AA | SFAMSWVRQAPGEGLEWVAIKISPGGSYTYY |
| | | | SDTVTGRFTISRDNAKNSLYLQMNSLRAED |
| | | | TAVYYCARQLWGYYALDIWGQGTTVTVSS |
| 127 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCCAGTTTAGTAGC |
| | | | TTTGCCATGTCTTGGGTCCGCCAGGCTCCAGGGAAGGGGCTGGAGTGGGT |
| | | | GGCCAAAATTAGTCCCGGTGGGAGTTACACCTACTATAGCGACACTGTGA |
| | | | CGGGCCGATTCACCATCTCCAGAGACAACGCCAAGAACTCACTGTATCTG |
| | | | CAAATGAACAGCCTGAGAGCCGAGGACACGGCTGTGTATTACTGTGCGAG |
| | | | ACAGTTATGGGGGTACTATGCTCTTGACATTTGGGGCCAAGGGACCACGG |
| | | | TCACCGTCTCCTCA |
| 128 | AME-22a | L Chain AA | EIVLTQSPATLSLSPGERATLSCSASYSVS |
| | | | YMYWYQQIKPGQAPRLLIYDFSNLASGIPAR |
| | | | FSGSGSGTDFTLTISSLEPEDFAVYYCMQW |
| | | | SGYPYTEGGGTKVE1K |
| 129 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCTACAGTGTAAGT |
| | | | TACATGTACTGGTACCAACAGAAACCTGGCCAGGCTCCCAGGCTCCTCAT |
| | | | CTATGACTTCTCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTATGCAGTGGAGTGGTTACCCATACACGTT |
| | | | CGGCGGAGGGACCAAGGTGGAGATCAAA |
| 130 | | H Chain | EVQLVESGGGLVQPGGSLRLSCAASGFQFS |
| | | AA | PFAMSWVRQAPGEGLEWVAIKISPGGSWTYY |
| | | | PDTDTGRFTISRDNAKNSLYLQMNSLRAED |
| | | | TAVYYCARQLWGYYALDFWGQGTTVTVSS |
| 131 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCCAGTTTAGTCCC |
| | | | TTTGCCATGTCTTGGGTCCGCCAGGCTCCAGGGAAGGGGCTGGAGTGGGT |
| | | | GGCCAAAATTAGTCCCGGTGGGAGTTGGACCTACTATCCTGACACTGACA |
| | | | CGGGCCGATTCACCATCTCCAGAGACAACGCCAAGAACTCACTGTATCTG |
| | | | CAAATGAACAGCCTGAGAGCCGAGGACACGGCTGTGSA?TACTGTGCGAG |
| | | | ACAGTTATGGGGGTACTATGCTCTTGACTTCTGGGGCCAAGGGACCACGG |
| | | | TCACCGTCTCCTCA |

TABLE 7
Amino acid sequence of a human light chain framework
region L6 with interspersed CDR sequences labeled

(FRL1-SEQ ID NO: 105) CDRL1 (FRL2-SEQ ID NO: 106) CDRL2
EIVLIQSPATLSLSPGERATLSCXXXXXXXXXXTNYQQKPGQAPRLLIYXXXXXXX
(FRL3-SEQ ID NO: 107) CDRL3 (FRL4-SEQ ID NO: 108)
GIPARFSGSGSGTDFTLTISSLEPEDFAVYYCXXXXXXXXXFGGGTKVEIK

TABLE 8
Amino acid sequence of a human heavy chain framework region
VH3-7 with interspersed CDR sequences labeled

(ERNI-SEQ ID NO: 109) CDRH1 (FRH2-SEQ ID NO: 110)
EVQLVESGGGLVQPGGSLRLSCAASXXXXXXXXXXWVRQAPGKGLEWVA
CDRH2 (FRH3-SEQ ID NO: 111)
XXXXXXXXXXXXXXXXXRFTISRDNAKNSLYLQMNSLRAEDTAVYYCAR
CDRH3 (FRH4-SEQ ID NO: 112)
XXXXXXXXXXWGQGTTVTVSS

TABLE 9
CDR Sequences

| SEQ | | |
|---|---|---|
| ID NO: | CDR | AA Sequence* |
| 132 | CDRL1 | SX₁X₂X₃X₄VX₅YMY |
| 133 | CDRL2 | DX₆SX₇LX₈S |
| 134 | CDRL3 | X₉X₁₀WSGYPYT |
| 135 | CDRH1 | GFX₁₁X₁₂SX₁₃FAX₁₄S |
| 136 | CDRH2 | KX₁₅SX₁₆GGSX₁₇X₁₈YX₁₉X₂₀DTX₂₁X₂₂X₂₃ |
| 137 | CDRH3 | QLWGX₂₄YALDX₂₅ |

SEQ ID NO: 115-AMINO ACID SEQUENCE OF IL-6 PROTEIN
MNSFSTSAFGPVAFSLGLLLVLPAAFPAPVPPGEDSKDVAAPHRQPLTS
SERIDKQIRYILDGISALRKETCNKSNMCESSKEALAENNLNLPKMAEK
DGCFQSGFNEETCLVKIITGLLEFEVYLEYLQNRFESSEEQARAVQMST
KVLIQFLQKKAKNLDAITTPDPTTNASLLTKLQAQNQWLQDMTTHLILR
SFKEFLQSSLRALRQM
*X denotes any suitable amino acid with exemplary, non-limiting amino acid substitutions shown in the sequences disclosed in SEQ ID NOS: 1-92 of Tables 1 and 2 and in Tables 3, 4, and 5A. In addition, X can have the following values:
X₁ = A or G
X₂ = S or R
X₃ = H, I, S, or Y
X₄ = S or Y
X₅ = S or F
X₆ = F, L, M, or T
X₇ = N or E
X₈ = A or T
X₉ = M, C, or S
X₁₀ = Q or C
X₁₁ = T or Q
X₁₂ = F, S, or T
X₁₃ = S or P
X₁₄ = L or M
X₁₅ = A or I
X₁₆ = S or P

An anti-IL-6 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, or as shown in SEQ ID NOS: 105-112, further optionally comprising at least one substitution, insertion or deletion), a heavy chain or light chain constant region, (e.g., comprising at least one CH1, hinge1, hinge2, hinge3, hinge4, CH2, or CH3 or fragment thereof, further optionally comprising at least one substitution, insertion or deletion), or any portion thereof, that can be incorporated into an antibody of the present invention. An antibody of the invention can include or be derived from any mammal, such as but not limited to, a human, a mouse, a rabbit, a rat, a rodent, a primate, or any combination thereof, and the like.

The isolated antibodies of the present invention comprise the antibody amino acid sequences disclosed herein encoded by any suitable polynucleotide, or any isolated or prepared antibody. Preferably, the human antibody or antigen-binding fragment binds human IL-6 and, thereby, partially or substantially neutralizes at least one biological activity of the protein. An antibody, or specified portion or variant thereof, that partially or preferably substantially neutralizes at least one biological activity of at least one IL-6 protein or fragment can bind the protein or fragment and thereby inhibit activities mediated through the binding of IL-6 to the IL-6 receptor or through other IL-6-dependent or mediated mechanisms. As used herein, the term “neutralizing antibody” refers to an antibody that can inhibit an IL-6-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-6 antibody to inhibit an IL-6-dependent activity is preferably assessed by at least one suitable IL-6 protein or receptor assay, as described herein and/or as known in the art. A human antibody of the invention can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, or γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the anti-human IL-6 human antibody comprises an IgG1 heavy chain and an IgG1 light chain.

Generally, the human antibody or antigen-binding fragment of the present invention will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original mouse CDRs can be used. These CDRs may be formed by incorporation of conservative substitutions from the original mouse sequence. As a non-limiting example, the antibody or antigen-binding portion or variant can comprise at least one of the heavy chain CDR3 having an amino acid sequence selected from the group consisting of SEQ ID NOS: 79, 81, 83, 85, 87, 89, and 91, and/or a light chain CDR3 having an amino acid sequence selected from the group consisting of SEQ ID NOS:29, 31, 33, and 35. In a particular embodiment, the antibody or antigen-binding fragment can have an antigen-binding region that comprises at least a portion of at least one heavy chain CDR (i.e., CDR1, CDR2 and/or CDR3) having the amino acid sequence of the corresponding CDRs 1, 2, and/or 3 (e.g., SEQ ID NOS:37, 49, and 79). In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3 (e.g., SEQ ID NOS:1, 17, and 29).

At least one antibody of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

### Purification of an Antibody

An anti-IL-6 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Cloning and Expression in CHO Cells

The vector pC4 is used for the expression of IL-6 antibody. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the IL-6 in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete IL-6 antibody is used, e.g., as presented in SEQ ID NOS: 7, and 8, corresponding to HC and LC variable regions of a IL-6 antibody of the present invention, according to known method steps. Isolated nucleic acid encoding a suitable human constant region (i.e., HC and LC regions) is also used in this construct.

The isolated variable and constant region encoding DNA and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

### Amino Acid Codes

The amino acids that make up anti-IL-6 antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994)

An anti-IL-6 antibody of the present invention can include one or more amino acid substitutions, deletions or additions, either from natural mutations or human manipulation, as specified herein. Amino acids in an anti-IL-6 antibody of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL-6 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Non-limiting variants that can enhance or maintain at least one of the listed activities include, but are not limited to, any of the above polypeptides, further comprising at least one mutation corresponding to at least one substitution in the residues varied among the disclosed variant amino acid sequences.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments of the invention can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-49-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

### Antibody Compositions Comprising Further Therapeutically Active Ingredients

The present invention also provides at least one anti-IL-6 antibody composition comprising at least one, at least two, at least three, at least four, at least five, at least six or more anti-IL-6 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL-6 antibody amino acid sequence selected from the group consisting of 70-100% of the contiguous amino acids of any of the antibody sequence disclosed herein, for example, SEQ ID NOS:1-92, or specified fragments, domains or variants thereof. Preferred anti-IL-6 antibody compositions include at least one or two full length, fragments, domains or variants as at least one CDR or LBR containing portions of the anti-IL-6 antibody sequence of 70-100% of, for example, SEQ ID NOS:1-92, or specified fragments, domains or variants thereof. Further preferred compositions comprise 40-99% of at least one of 70-100% of SEQ ID NOS:1-92, or specified fragments, domains or variants thereof. Such composition percentages are by weight, volume, concentration, molarity, or molality as liquid or dry solutions, mixtures, suspension, emulsions or colloids, as known in the art or as described herein.

The antibody compositions of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21st edition, Springhouse Corp., Springhouse, P A, 2001; Health Professional's Drug Guide 2001, ed., Shannon, Wilson, Stang, Prentice-Hall, Inc, Upper Saddle River, N.J.; Pharmcotherapy Handbook, Wells et al., ed., Appleton & Lange, Stamford, Conn., each entirely incorporated herein by reference).

The CNS drug can be at least one selected from nonnarcotic analgesics or at least one selected from antipyretics, nonsteroidal anti-inflammatory drugs, narcotic or at least one opioid analgesics, sedative-hypnotics, anticonvulsants, antidepressants, antianxiety drugs, antipsychotics, central nervous system stimulants, antiparkinsonians, and miscellaneous central nervous system drugs. The ANS drug can be at least one selected from cholinergics (parasympathomimetics), anticholinergics, adrenergics (sympathomimetics), adrenergic blockers (sympatholytics), skeletal muscle relaxants, and neuromuscular blockers. The respiratory tract drug can be at least one selected from antihistamines, bronchodilators, expectorants or at least one antitussive, and miscellaneous respiratory drugs. The GI tract drug can be at least one selected from antacids or at least one adsorbent or at least one antiflatulent, digestive enzyme or at least one gallstone solubilizer, antidiarrheals, laxatives, antiemetics, and antiulcer drugs. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The immunomodulation drug can be at least one selected from immunosuppressants, vaccines or at least one toxoid, antitoxin or at least one antivenin, immune serum, and biological response modifier. The ophthalmic, otic, and nasal drugs can be at least one selected from ophthalmic anti-infectives, ophthalmic anti-inflammatories, miotics, mydriatics, ophthalmic vasoconstrictors, miscellaneous ophthalmics, otics, and nasal drugs. See, e.g., contents of Nursing 2001 Drug Handbook, supra.

The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef. (See, e.g., pp. 24-214 of Nursing 2001 Drug Handbook.)

The at least one nonnarcotic analgesic or antipyretic can be at least one selected from acetaminophen, aspirin, choline magnesium trisalicylate, diflunisal, and magnesium salicylate. The at least one nonsteroidal anti-inflammatory drug can be at least one selected from celecoxib, diclofenac potassium, diclofenac sodium, etodolac, fenoprofen calcium, flurbiprofen, ibuprofen, indomethacin, indomethacin sodium trihydrate, ketoprofen, ketorolac tromethamine, nabumetone, naproxen, naproxen sodium, oxaprozin, piroxicam, rofecoxib, and sulindac. The at least one narcotic or opioid analgesic can be at least one selected from alfentanil hydrochloride, buprenorphine hydrochloride, butorphanol tartrate, codeine phosphate, codeine sulfate, fentanyl citrate, fentanyl transdermal system, fentanyl transmucosal, hydromorphone hydrochloride, meperidine hydrochloride, methadone hydrochloride, morphine hydrochloride, morphine sulfate, morphine tartrate, nalbuphine hydrochloride, oxycodone hydrochloride, oxycodone pectinate, oxymorphone hydrochloride, pentazocine hydrochloride, pentazocine hydrochloride and naloxone hydrochloride, pentazocine lactate, propoxyphene hydrochloride, propoxyphene napsylate, remifentanil hydrochloride, sufentanil citrate, and tramadol hydrochloride. The at least one sedative-hypnotic can be at least one selected from chloral hydrate, estazolam, flurazepam hydrochloride, pentobarbital, pentobarbital sodium, phenobarbital sodium, secobarbital sodium, temazepam, triazolam, zaleplon, and zolpidem tartrate. The at least one anticonvulsant can be at least one selected from acetazolamide sodium, carbamazepine, clonazepam, clorazepate dipotassium, diazepam, divalproex sodium, ethosuximde, fosphenytoin sodium, gabapentin, lamotrigine, magnesium sulfate, phenobarbital, phenobarbital sodium, phenytoin, phenytoin sodium, phenytoin sodium (extended), primidone, tiagabine hydrochloride, topiramate, valproate sodium, and valproic acid. The at least one antidepressant can be at least one selected from amitriptyline hydrochloride, amitriptyline pamoate, amoxapine, bupropion hydrochloride, citalopram hydrobromide, clomipramine hydrochloride, desipramine hydrochloride, doxepin hydrochloride, fluoxetine hydrochloride, imipramine hydrochloride, imipramine pamoate, mirtazapine, nefazodone hydrochloride, nortriptyline hydrochloride, paroxetine hydrochloride, phenelzine sulfate, sertraline hydrochloride, tranylcypromine sulfate, trimipramine maleate, and venlafaxine hydrochloride. The at least one antianxiety drug can be at least one selected from alprazolam, buspirone hydrochloride, chlordiazepoxide, chlordiazepoxide hydrochloride, clorazepate dipotassium, diazepam, doxepin hydrochloride, hydroxyzine embonate, hydroxyzine hydrochloride, hydroxyzine pamoate, lorazepam, mephrobamate, midazolam hydrochloride, and oxazepam. The at least one antipsychotic drug can be at least one selected from chlorpromazine hydrochloride, clozapine, fluphenazine decanoate, fluephenazine enanthate, fluphenazine hydrochloride, haloperidol, haloperidol decanoate, haloperidol lactate, loxapine hydrochloride, loxapine succinate, mesoridazine besylate, molindone hydrochloride, olanzapine, perphenazine, pimozide, prochlorperazine, quetiapine fumarate, risperidone, thioridazine hydrochloride, thiothixene, thiothixene hydrochloride, and trifluoperazine hydrochloride. The at least one central nervous system stimulant can be at least one selected from amphetamine sulfate, caffeine, dextroamphetamine sulfate, doxapram hydrochloride, methamphetamine hydrochloride, methylphenidate hydrochloride, modafinil, pemoline, and phentermine hydrochloride. The at least one antiparkinsonian can be at least one selected from amantadine hydrochloride, benztropine mesylate, biperiden hydrochloride, biperiden lactate, bromocriptine mesylate, carbidopa-levodopa, entacapone, levodopa, pergolide mesylate, pramipexole dihydrochloride, ropinirole hydrochloride, selegiline hydrochloride, tolcapone, and trihexyphenidyl hydrochloride. The at least one miscellaneous central nervous system drug can be at least one selected from bupropion hydrochloride, donepezil hydrochloride, droperidol, fluvoxamine maleate, lithium carbonate, lithium citrate, naratriptan hydrochloride, nicotine polacrilex, nicotine transdermal system, propofol, rizatriptan benzoate, sibutramine hydrochloride monohydrate, sumatriptan succinate, tacrine hydrochloride, and zolmitriptan. (See, e.g., pp. 337-530 of Nursing 2001 Drug Handbook.)

The at least one cholinergic (e.g., parasymathomimetic) can be at least one selected from bethanechol chloride, edrophonium chloride, neostigmine bromide, neostigmine methylsulfate, physostigmine salicylate, and pyridostigmine bromide. The at least one anticholinergic can be at least one selected from atropine sulfate, dicyclomine hydrochloride, glycopyrrolate, hyoscyamine, hyoscyamine sulfate, propantheline bromide, scopolamine, scopolamine butylbromide, and scopolamine hydrobromide. The at least one adrenergic (sympathomimetics) can be at least one selected from dobutamine hydrochloride, dopamine hydrochloride, metaraminol bitartrate, norepinephrine bitartrate, phenylephrine hydrochloride, pseudoephedrine hydrochloride, and pseudoephedrine sulfate. The at least one adrenergic blocker (sympatholytic) can be at least one selected from dihydroergotamine mesylate, ergotamine tartrate, methysergide maleate, and propranolol hydrochloride. The at least one skeletal muscle relaxant can be at least one selected from baclofen, carisoprodol, chlorzoxazone, cyclobenzaprine hydrochloride, dantrolene sodium, methocarbamol, and tizanidine hydrochloride. The at least one neuromuscular blocker can be at least one selected from atracurium besylate, cisatracurium besylate, doxacurium chloride, mivacurium chloride, pancuronium bromide, pipecuronium bromide, rapacuronium bromide, rocuronium bromide, succinylcholine chloride, tubocurarine chloride, and vecuronium bromide. (See, e.g., pp. 531-84 of Nursing 2001 Drug Handbook.)

The at least one corticosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, and tacrolimus. The at least one biological response modifier can be at least one selected from aldesleukin, epoetin alfa, filgrastim, glatiramer acetate for injection, interferon alfacon-1, interferon alfa-2a (recombinant), interferon alfa-2b (recombinant), interferon beta-1a, interferon beta-1b (recombinant), interferon gamma-1b, levamisole hydrochloride, oprelvekin, and sargramostim. (See, e.g., pp. 964-1040 of Nursing 2001 Drug Handbook.)

The at least one nasal drug can be at least one selected from beclomethasone dipropionate, budesonide, ephedrine sulfate, epinephrine hydrochloride, flunisolide, fluticasone propionate, naphazoline hydrochloride, oxymetazoline hydrochloride, phenylephrine hydrochloride, tetrahydrozoline hydrochloride, triamcinolone acetonide, and xylometazoline hydrochloride. (See, e.g., pp. 1041-97 of Nursing 2001 Drug Handbook.)

For example, the at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (See, e.g., pp. 1098-1136 of Nursing 2001 Drug Handbook.)

Anti-IL-6 antibody compositions of the present invention can further comprise at least one of any suitable and effective amount of a composition or pharmaceutical composition comprising at least one anti-IL-6 antibody contacted or administered to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, etanercept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Non-limiting examples of such cytokines include, but are not limited to, any of IL-1 to IL-23 (e.g., IL-1, IL-2, etc.). Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Anti-IL-6 antibody compounds, compositions or combinations of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the anti-IL-6 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-6 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-6 antibody compositions of the invention can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-6 antibody, portion or variant compositions according to the invention are known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

### Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-6 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used as known in the art, such as about 0.0015%, or any range, value, or fraction therein. Non-limiting examples include, no preservative, about 0.1-2% m-cresol (e.g., 0.2, 0.3. 0.4, 0.5, 0.9, 1.0%), about 0.1-3% benzyl alcohol (e.g., 0.5, 0.9, 1.1, 1.5, 1.9, 2.0, 2.5%), about 0.001-0.5% thimerosal (e.g., 0.005, 0.01), about 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one anti-IL-6 antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one anti-IL-6 antibody, and a second vial comprising an aqueous diluent of prescribed buffer or preservative, wherein said packaging material comprises a label that instructs a patient to reconstitute the at least one anti-IL-6 antibody in the aqueous diluent to form a solution that can be held over a period of twenty-four hours or greater.

The at least one anti-IL-6 antibody used in accordance with the present invention can be produced by recombinant means, including from mammalian cell or transgenic preparations, or can be purified from other biological sources, as described herein or as known in the art.

The range of at least one anti-IL-6 antibody in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one anti-IL-6 antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-6 antibody and preservative in an aqueous diluent is carried out using conventional dissolution and mixing procedures. To prepare a suitable formulation, for example, a measured amount of at least one anti-IL-6 antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed formulations can be provided to patients as clear solutions or as dual vials comprising a vial of lyophilized at least one anti-IL-6 antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biological activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one anti-IL-6 antibody of the invention can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-IL-6 antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-IL-6 antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www. mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors and needle free IV infusion sets.

The products presently claimed include packaging material. The packaging material provides, in addition to the information required by the regulatory agencies, the conditions under which the product can be used. The packaging material of the present invention provides instructions to the patient to reconstitute the at least one anti-IL-6 antibody in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one anti-IL-6 antibody and a selected buffer, preferably, a phosphate buffer containing saline or a chosen salt. Mixing the at least one anti-IL-6 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one anti-IL-6 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-6 antibody may result in other than a clear solution of lyophilized powder comprising the antibody. Among non-clear solutions are formulations comprising particulate suspensions, said particulates being a composition containing the anti-IL-6 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gelatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(β-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

At least one anti-IL-6 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Alternative Administration

Many known and developed modes can be used according to the present invention for administering pharmaceutically effective amounts of at least one anti-IL-6 antibody according to the present invention. While pulmonary administration is used in the following description, other modes of administration can be used according to the present invention with suitable results. IL-6 antibodies of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of at least one anti-IL-6 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. At least one anti-IL-6 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

### Pulmonary/Nasal Administration

For pulmonary administration, preferably, at least one anti-IL-6 antibody composition is delivered in a particle size effective for reaching the lower airways of the lung or sinuses. According to the invention, at least one anti-IL-6 antibody can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of antibodies are also known in the art. All such devices can use formulations suitable for the administration for the dispensing of antibody in an aerosol. Such aerosols can be comprised of either solutions (both aqueous and non-aqueous) or solid particles.

Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention.

Preferably, a composition comprising at least one anti-IL-6 antibody is delivered by a dry powder inhaler or a sprayer. There are several desirable features of an inhalation device for administering at least one antibody of the present invention. For example, delivery by the inhalation device is advantageously reliable, reproducible, and accurate. The inhalation device can optionally deliver small dry particles, e.g., less than about 10 μm, preferably about 1-5 μm, for good respirability.

### Administration of IL-6 Antibody Compositions as a Spray

A spray including IL-6 antibody composition can be produced by forcing a suspension or solution of at least one anti-IL-6 antibody through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one anti-IL-6 antibody composition delivered by a sprayer have a particle size less than about 10 μm, preferably, in the range of about 1 μm to about 5 μm, and, most preferably, about 2 μm to about 3 μm.

Formulations of at least one anti-IL-6 antibody composition suitable for use with a sprayer typically include antibody composition in an aqueous solution at a concentration of about 0.1 mg to about 100 mg of at least one anti-IL-6 antibody composition per ml of solution or mg/gm, or any range, value, or fraction therein. The formulation can include agents, such as an excipient, a buffer, an isotonicity agent, a preservative, a surfactant, and, preferably, zinc. The formulation can also include an excipient or agent for stabilization of the antibody composition, such as a buffer, a reducing agent, a bulk protein, or a carbohydrate. Bulk proteins useful in formulating antibody compositions include albumin, protamine, or the like. Typical carbohydrates useful in formulating antibody compositions include sucrose, mannitol, lactose, trehalose, glucose, or the like. The antibody composition formulation can also include a surfactant, which can reduce or prevent surface-induced aggregation of the antibody composition caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein, such as IL-6 antibodies, or specified portions or variants, can also be included in the formulation.

### Oral Formulations and Administration

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and antibodies and a combination of at least two surfactants intended for oral, buccal, mucosal, nasal, pulmonary, vaginal transmembrane, or rectal administration are taught in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 and U.S. Pat. No. 5,871,753 and used to deliver biologically active agents orally are known in the art.

### Mucosal Formulations and Administration

A formulation for orally administering a bioactive agent encapsulated in one or more biocompatible polymer or copolymer excipients, preferably, a biodegradable polymer or copolymer, affording microcapsules which due to the proper size of the resultant microcapsules results in the agent reaching and being taken up by the folliculi lymphatic aggregati, otherwise known as the “Peyer's patch,” or “GALT” of the animal without loss of effectiveness due to the agent having passed through the gastrointestinal tract. Similar folliculi lymphatic aggregati can be found in the bronchei tubes (BALT) and the large intestine. The above-described tissues are referred to in general as mucosally associated lymphoreticular tissues (MALT). For absorption through mucosal surfaces, compositions and methods of administering at least one anti-IL-6 antibody include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695).

### Transdermal Formulations and Administration

For transdermal administration, the at least one anti-IL-6 antibody is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599).

### Prolonged Administration and Formulations

It can be desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the compounds of the present invention or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Clinical Experience with Anti-IL-6 Agents

Several clinical trials using monoclonal antibodies against IL-6 have been conducted in multiple diseases including plasma cell leukemia, multiple myeloma, B-lympho-proliferative disorder, rheumatoid arthritis, renal carcinoma, and AIDS associated lymphoma.

A Phase I dose escalating study with the anti-IL-6 cCLB-8 antibody of the present invention for the treatment of refractory patients with advanced stage multiple myeloma (N=12) demonstrated that some patients had disease stabilization. After discontinuation of treatment there was acceleration in the increase of M protein levels, suggesting disease re-bound after the withdrawal of therapy. Anti-IL-6 cCLB-8 antibody inhibited free circulating IL-6. Most importantly no toxicity (except transient thrombocytopenia in two heavily pretreated patients) or allergic reactions were observed. C-reactive protein (CRP) decreased below detection level in all patients. Anti-IL-6 cCLB-8 antibody demonstrated a long circulating half-life of 17.8 days, and there was no human anti-chimeric antibody (HACA) immune response observed (van Zaanen et al. 1998). Administration of CNTO 328 did not cause changes in blood pressure, pulse rate, temperature, hemoglobin, liver functions and renal functions. Except for transient thrombocytopenia in two heavily pretreated patients, no toxicity or allergic reactions were observed, and there was no human anti-chimeric antibody (HACA) immune response observed. Three patients developed infection-related complications during therapy, however, a possible relation with anti-IL-6 cCLB-8 antibody was unlikely because infectious complications are common in end stage multiple myeloma and are a major cause of death. In addition all three patients were able to respond to their infection even in the presence of anti-IL-6 cCLB-8 antibody, suggesting that anti-IL-6 therapy is not able to block IL-6 during infection. No treatment-associated fatalities were reported. In conclusion, results from this study suggest that anti-IL-6 cCLB-8 antibody was safe in multiple myeloma patients.

Thus, the present invention provides a method for modulating or treating at least one malignant disease in a cell, tissue, organ, animal or patient, including, but not limited to, at least one of: multiple myeloma, leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, renal cell carcinoma, pancreatic carcinoma, prostatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, cancer related bone resorption, cancer related bone pain; the suppression of cancer metastasis; the amelioration of cancer cachexia; and the treatment of inflammatory diseases such as mesangial proliferative glomerulonephritis and the like. Such a method can optionally be used in combination with, by administering before, concurrently or after administration of such IL-6 antibody, radiation therapy, an anti-angiogenic agent, a chemotherapeutic agent, a farnesyl transferase inhibitor or the like.

The present invention also provides a method for modulating or treating at least one 11-6 mediated immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, asteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitivity reactions, allergic rhinitis, hay fever, perennial rhinitis, conjunctivitis, endometriosis, asthma, urticaria, systemic anaphalaxis, dermatitis, pernicious anemia, hemolytic disease, thrombocytopenia, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited to asthenia, anemia, cachexia, and the like), chronic salicylate intoxication, sleep apnea, obesity, heart failure, sinusitis, inflammatory bowel disease, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

The present invention also provides a method for modulating or treating at least one infectious disease in a cell, tissue, organ, animal or patient, including, but not limited to, at least one of: acute or chronic bacterial infection, acute and chronic parasitic or infectious processes, including bacterial, viral and fungal infections, HIV infection/HIV neuropathy, meningitis, hepatitis (A, B or C, or the like), septic arthritis, peritonitis, pneumonia, epiglottitis, e. coli 0157:h7, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, vital-associated hemaphagocytic syndrome, vital encephalitis/aseptic meningitis, and the like;

Any of such methods can optionally comprise administering an effective amount of at least one composition or pharmaceutical composition comprising at least one anti-IL-6 antibody to a cell, tissue, organ, animal or patient in need of such modulation, treatment or therapy. Indications for treatment with ant-IL-6 therapy are disclosed in the following references, hereby incorporated by reference into the present application: Van Snick, “Interleukin-6: An Overview,” Ann. Rev. Immunol., 8:253-278 (1990); Campbell et al., “Essential Role for Interferon-gamma. And Interleukin-6 in Autoimmune Insulin-Dependent Diabetes in NOD/Wehi Mice,” J. Clin. Invest., 87:739-742 (1991); Heinrich et al., “Interleukin-6 Monoclonal Antibody Therapy for a Patient with Plasma Cell Leukemia,” Blood, 78(5):1198-1204 (1991); Starnes et al., “Anti-IL-6 Monoclonal Antibodies Protect Against Lethal Escherichia coli Infection and Lethal Tumor Necrosis Factor-alpha. Challenge in Mice,” J. Immunol., 145(12):4185-4191 (1990); Strassman et al., “Evidence for the Involvement of Interleukin 6 in Experimental Cancer Cachexia,” J. Clin. Invest., 89:1681-1684 (1992).

Any method of the present invention can comprise administering an effective amount of a composition or pharmaceutical composition comprising at least one anti-IL-6 antibody to a cell, tissue, organ, animal or patient in need of such modulation, treatment or therapy. Such a method can optionally further comprise co-administration or combination therapy for treating such immune diseases or malignant diseases, wherein the administering of said at least one anti-IL-6 antibody, specified portion or variant thereof, further comprises administering, before concurrently, and/or after, at least one selected from at least one TNF antagonist (e.g., but not limited to a TNF antibody or fragment, a soluble TNF receptor or fragment, fusion proteins thereof, or a small molecule TNF antagonist), an IL-18 antibody or fragment, small molecule IL-18 antagonist or IL-18 receptor binding protein, an IL-1 antibody (including both IL-1 alpha and IL-1 beta) or fragment, a soluble IL-1 receptor antagonist, an antirheumatic (e.g., methotrexate, auranofin, aurothioglucose, azathioprine, etanercept, gold sodium thiomalate, hydroxychloroquine sulfate, leflunomide, sulfasalazine, radiation therapy, an anti-angiogenic agent, a chemotherapeutic agent, Thalidomide, a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteroid, an anabolic steroid, a diabetes related agent, a mineral, a nutritional, a thyroid agent, a vitamin, a calcium related hormone, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

The following examples illustrate the invention. These examples should not be construed as to limit the scope of this invention. The examples are included for purposes of illustration and the present invention is limited only by the claims.

### Example 1: Clinical Trials

The sirukumab data set is from a phase II randomized, double-blind, placebo controlled clinical trial, NCT00718718 in MTX resistant RA patients with screening CRP≧10 mg/L (Smolen, J. S., et al., Ann Rheum Dis 73:1616-1625 (2014)). Treatment was administered subcutaneously (100 mg every 2 weeks, or 25, 50 or 100 mg every 4 weeks). All dosing arms were combined for analyses shown here to maximize sample size. Eleven subjects reporting usage of anti-depressant medications were excluded from the analysis. 176 RA patients were included in the analysis of baseline statistics, with exact sample sizes for each analysis are shown in figures, accounting for missing data due to dropouts or missed visits. RA Symptom severity was measured using the Disease Activity Score using C-Reactive Protein, DAS28-CRP (Hulsey, T. C., et al., J S C Med Assoc 85:357-384 (1989)). RA responder status was determined using the American College of Rheumatology 50 response (ACR50) at Week 12, which corresponds to a 50% change in joint swelling and pain from baseline (Aletaha, D., et al., Arthritis Rheum 62:2569-2581 (2010)).

The siltuximab data set is from a phase 2 randomized, double-blind, placebo controlled clinical trial (NCT01024036) in HPV8-negative MCD patients with no prior exposure to IL-6 or IL-6R targeted therapies (van Rhee, F., et al., Lancet Oncol 15:966-974 (2014)). A subset of patients were treated with corticosteroids (15 in siltuximab treated group, 8 in placebo group), not exceeding a dose of 1 mg/kg/day of prednisone, that remained stable or decreased during the 4 weeks prior to study start. Ultimately 79 subjects were treated with siltuximab and included in our analysis. Symptom severity was captured using the Multicentric Castleman's Disease Signs and Symptoms Score (van Rhee, F., et al., Patient 8:207-216 (2015)).

The first follow up visit was assessed in each study: week 12 for sirukumab, and week 6 for siltuximab. Cytokine biomarkers were evaluated for correlations with depressive symptom improvement where available.

### Serum Biomarkers

In the sirukumab study, serum samples were separated and stored at −80° C. until further processing. ELISA assay were performed by Quintiles Labs for high-sensitivity C-Reactive Protein (CRP) and Serum Amyloid A (SAA). IL-6 assays were performed using the Mesoscale Diagnostics (MSD) Ultra-sensitive kit (K111AKC). sIL6R ELISA assays were performed using R&D Systems (DR600), and sgp130 assays using R&D Systems (DGP00). In the siltuximab study, CRP samples were analyzed at Covance using a high sensitivity CRP assay with lower limit of quantification (LLOQ) of 0.20 mg/L.

### Quantification of Depressive Symptoms

Two core depressive symptoms (depressed mood and anhedonia) and two fatigue symptoms (worn out and tired) were documented on the SF-36 Health Survey, version 2.0 (Ware J. E. et al., QualityMetric (2001)). Patients were grouped by presence/absence of prevalent depressed mood and anhedonia (PDMA), meaning one of the depressive symptoms was present at least ‘most of the time’ and the other at least ‘some of the time’ for four weeks. Treatment groups were defined as receiving any treatment dose.

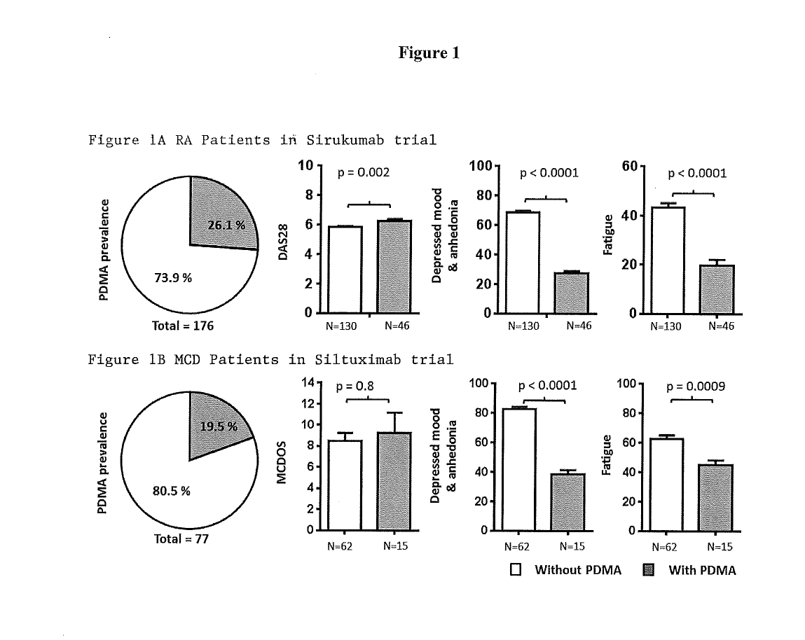

In both trials, the 36-item short form health survey (SF-36) questionnaire (version 2.0) was used (Ware J. E. et al., QualityMetric (2001)). Previously, it was shown that the mental health component score of the SF-36 returns to population norms in the Siltuximab study. However within this score, based on 14 items, not all items are related specifically to depressive symptoms, which we are interested in here. Two core depressive symptoms (depressed mood and anhedonia) and two fatigue symptoms (worn out and tired) were documented and continuous depressed mood and fatigue scores were created by summing the scores for the two respective questions, and scaling from 0 to100. Patients were grouped by presence/absence of prevalent depressed mood and anhedonia (PDMA) at baseline, meaning one of the depressive symptoms was present at least ‘most of the time’ and the other at least ‘some of the time’ in the previous 4 weeks (FIGS. 1A and 1B).

### Analysis and Statistics

Comparisons were made between patient groups with and without PDMA at baseline for baseline clinical measures, demographics, and baseline levels of serum biomarkers using either chi-square tests or Wilcoxon two-sample rank sum tests when appropriate. Correlations with baseline serum biomarkers were assessed using (partial) Spearman correlation coefficients. Treatment effects were examined using mixed models with repeated measures (MMRMs). Separate models were fit for patients with and without PDMA at baseline. Dependent variables were depressive symptom scores. Treatment, visit, and treatment-by-visit interaction were fixed effects with visit included as a repeated measure. For the siltuximab study, corticosteroid use is included as an additional fixed effect. To account for changes in RA or MCD symptoms, the DAS28-CRP or MCDOS (respectively) was added as a time-dependent fixed effect. The within-treatment-arm improvement was estimated by contrasting the least square means of depressive symptom score at pre- and post-treatment visit in the corresponding treatment arm. The treatment effect was estimated by contrasting the improvement in the treated arm and the improvement in the placebo arm. A p-value threshold of 0.05 was used to declare statistical significance. All analyses were performed using SAS 9.2

### Example 2: Patient Characteristics and Serum Biomarkers at Trial Entry

Prevalent depressed mood and anhedonia (PDMA) is experienced at baseline by 46 (26%) of the 176 RA patients, and 15 (20%) of the 77 MCD patients (FIGS. 1A and 1B). The key demographic and baseline characteristics of the sirukumab and siltuximab patient cohorts with and without PDMA are shown in Table 11. Overall, there are no significant demographic differences with the exception of a slightly younger age among RA patients with PDMA, and significant difference in racial distribution.

Both the depressed mood and anhedonia and fatigue scores were significantly correlated with RA severity in the overall group at baseline (Table 10A). A slight but significantly higher RA severity is detected in patients with PDMA (Table 11, FIG. 1A). In contrast, neither score was significantly correlated with MCD severity in the overall group (Table 10B), and no significant difference in MCD severity is detected between patients with and without PDMA (Table 11, FIG. 1B). In both studies, patients with PDMA exhibited significantly more fatigue than those without PDMA (FIGS. 1A and 1B).

TABLE 10
Correlations between RA (A) or MCD (B) symptom
severity and depression related measures at trial entry.

| | All Patients | | | With PDMA | | | Without PDMA | | |
|---|---|---|---|---|---|---|---|---|---|
| SF36 items | n | Spearman r | p | n | Spearman r | p | n | Spearman r | p |

A

| | | | | | | | | | |
|---|---|---|---|---|---|---|---|---|---|
| Depressed mood | 176 | −0.28 | 1.6E−04 | 46 | −0.13 | 0.38 | 130 | −0.18 | 0.039 |
| and anhedonia | | | | | | | | | |
| Fatigue | 176 | −0.31 | 2.4E−05 | 46 | −0.16 | 0.29 | 130 | −0.23 | 0.007 |

B

| | | | | | | | | | |
|---|---|---|---|---|---|---|---|---|---|
| Depressed mood | 77 | −0.16 | 0.157 | 15 | −0.08 | 0.779 | 62 | −0.22 | 0.089 |
| and anhedonia | | | | | | | | | |
| Fatigue | 76 | −0.22 | 0.055 | 15 | 0.2 | 0.466 | 61 | −0.28 | 0.030 |

In RA patients, in the overall group at baseline, the DAS28-CRP was significantly correlated with levels of CRP and IL-6, but not sIL6R and sgp130 (Table 12A). No significant correlation between scores of depressed mood and anhedonia and levels of CRP, LI6, sIL6R and sgp130 was observed (Table 12A). No significant differences are observed in serum levels of CRP, IL-6, and sgp130 on stratification by PDMA; however, there is a trend towards higher sIL-6R levels in patients with PDMA (p=0.09; Table 11).

In MCD patients, in the overall group at baseline, no significant correlations were found between either the MCDDOS severity or depressed mood and anhedonia score with levels of CRP or IL-6 (Table 12B). Severity of fatigue was significantly correlated with levels of CRP, but not IL-6 (Table 12B). On stratification, MCD patients with PDMA exhibited significantly higher levels of CRP than patients without PDMA (p=0.03); no significant difference was seen in IL-6 (Table 11).

TABLE 11
Demographics, clinical characteristics, and biomarker levels at the trial entry.

| Sirukumab | | | | Siltuximab | | | |
|---|---|---|---|---|---|---|---|
| | | Without | | | With | Without | |
| All | With PDMA | PDMA | | All | PDMA | PDMA | |
| N = 176 | N = 46 | N = 130 | p values | N = 77 | N = 15 | N =62 | p values |

| Age (yr) | | 51.7 ± 11.1 | 49.3 ± 10.5 | 52.6 ± 11.2 | 0.045 | 45.3 ± 13.5 | 46.0 ± 13.4 | 45.1 ± 13.6 | 0.67 |
|---|---|---|---|---|---|---|---|---|---|
| Gender (Male, n, (%)) | | 33 (18.8) | 6 (13.0) | 27 (20.8) | 0.25 | 51 (66.2) | 9 (60.0) | 42 (67.7) | 0.56 |
| Race | Asian | 30 (17.0) | 3 (6.5) | 27 (20.8) | 0.03 | 38 (49.4) | 10 (66.7) | 28 (45.2) | 0.04 |
| n (%) | Black | 4 (2.3) | 1 (2.2) | 3 (2.3) | | 3 (3.9) | 2 (13.3) | 1 (1.6) | |
| | Caucasian | 118 (67.0) | 31 (67.4) | 87 (66.9) | | 29 (37.7) | 3 (20.0) | 26 (41.9) | |
| | Other | 24 (13.6) | 11 (23.9) | 13 (10) | | 7 (9.1) | 0 (0) | 7 (11.3) | |

| | | | | | | | | |
|---|---|---|---|---|---|---|---|---|
| RA duration (yr) | 8.0 ± 7.3 | 8.5 ± 6.9 | 7.8 ± 7.4 | 0.32 | — | — | — | — |
| RA severity | 6 ± 0.9 | 6.3 ± 0.8 | 5.8 ± 0.8 | 0.002 | — | — | — | — |
| (DAS28-CRP) | | | | | | | | |
| MCD severity | — | — | — | — | 8.6 ± 5.2 | 9.3 ± 7.4 | 8.5 ± 5.9 | 0.80 |
| (MCD-SS) | | | | | | | | |
| CRP, mg/L | 25.3 ± 22.0 | 26.2 ± 24.5 | 25.0 ± 21.1 | 0.9 | 36.3 ± 48.0 | 66.1 ± 62.6 | 29.1 ± 41.2 | 0.03 |
| IL6, pg/ml | 25.3 ± 56.7¹ | 25.6 ± 37.3 | 25.2 ± 62.3² | 0.52 | 8.0 ± 8.2 | 9.5 ± 0.76 | 7.6 ± 8.3 | 0.18 |
| sIL6R, ng/ml | 41.0 ± 13.1 | 44.3 ± 12.9 | 39.9 ± 13.0 | 0.09 | — | — | — | — |
| sgp130, ng/ml | 276.2 ± 63.9 | 282.4 ± 68.7 | 274 ± 62.2 | 0.35 | — | — | — | — |

Data collection and statistical analyses are described in the Methods. Age, disease duration and severity, and biomarkers are reported as mean ± SD.
P-values correspond to with PDMA vs. without PDMA comparison.
¹N = 93.
²N = 32.

TABLE 12
Correlations between clinical measures and levels of some
biomarkers at trial entry in patients with RA (A) and MCD (B)

| | | | Without RA | | With RA severity | |
|---|---|---|---|---|---|---|
| | Serum | | severity adjustment | | adjustment | |
| Clinical Variable | Biomarker | N | Spearman r | p-value | Spearman r | p-value |

A

| | | | | | | |
|---|---|---|---|---|---|---|
| RA severity (CRP-DAS28) | CRP | 176 | 0.43 | 3.5E−09 | | |
| | IL6 | 174 | 0.21 | 0.0048 | | |
| | sIL6R | 176 | −0.02 | 0.83 | | |
| | sgp130 | 176 | 0.00 | 1.00 | | |
| Depressed mood and | CRP | 176 | −0.05 | 0.50 | 0.08 | 0.30 |
| anhedonia | IL6 | 174 | 0.01 | 0.86 | 0.08 | 0.30 |
| | sIL6R | 176 | −0.11 | 0.15 | −0.12 | 0.12 |
| | sgp130 | 176 | −0.10 | 0.17 | −0.11 | 0.16 |
| Fatigue | CRP | 176 | −0.13 | 0.090 | 0.01 | 0.93 |
| | IL6 | 174 | −0.06 | 0.45 | 0.01 | 0.91 |
| | sIL6R | 176 | −0.12 | 0.13 | −0.13 | 0.093 |
| | sgp130 | 176 | −0.16 | 0.038 | −0.17 | 0.029 |

B

| | | | | | | |
|---|---|---|---|---|---|---|
| MCD severity (MCDOS) | CRP | 77 | −0.03 | 0.80 | | |
| | IL6 | 73 | 0.18 | 0.13 | | |
| Depressed mood and | CRP | 77 | −0.17 | 0.13 | −0.18 | 0.11 |
| anhedonia | IL6 | 73 | −0.15 | 0.20 | −0.13 | 0.28 |
| Fatigue | CRP | 76 | −0.26 | 0.026 | −0.27 | 0.017 |
| | IL6 | 72 | −0.2 | 0.09 | −0.17 | 0.16 |

### Example 3: Effects of IL-6 Neutralizing Antibodies on Outcome Measures of Primary Diseases

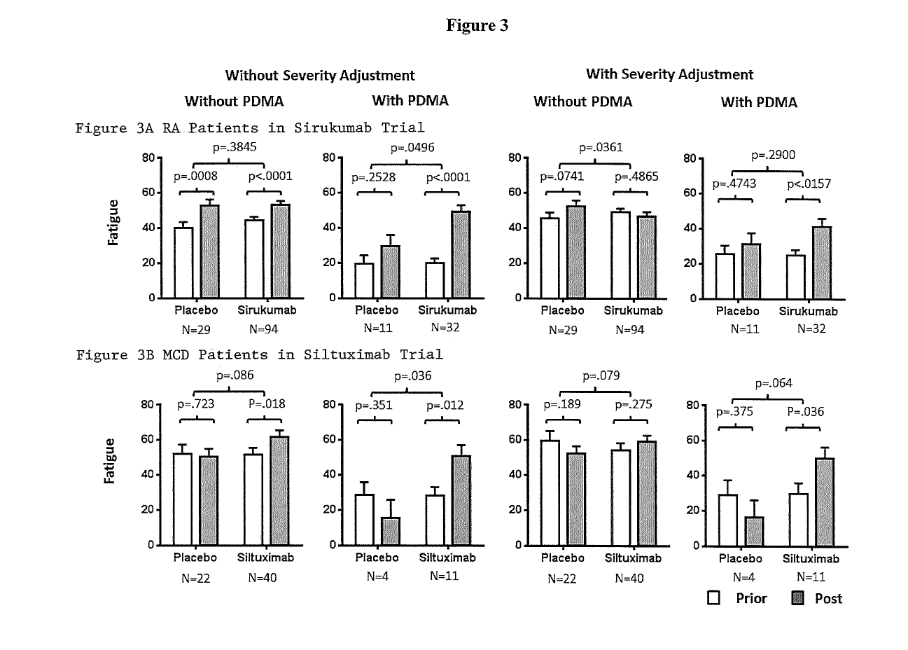

Sirukumab treatment significantly improved RA severity as assessed by the DAS28-CRP in subjects with and without PDMA and the clinical effect was also significantly better than placebo (FIG. 3A). Siltuximab treatment significantly improved MCD symptom severity as assessed by the MCDDOS in subjects with and without PDMA, but there is no significant difference from placebo arm at the 6 week time point (FIG. 3B).

### Example 4: Effect of IL-6 Neutralizing Antibodies on Depressive Symptoms

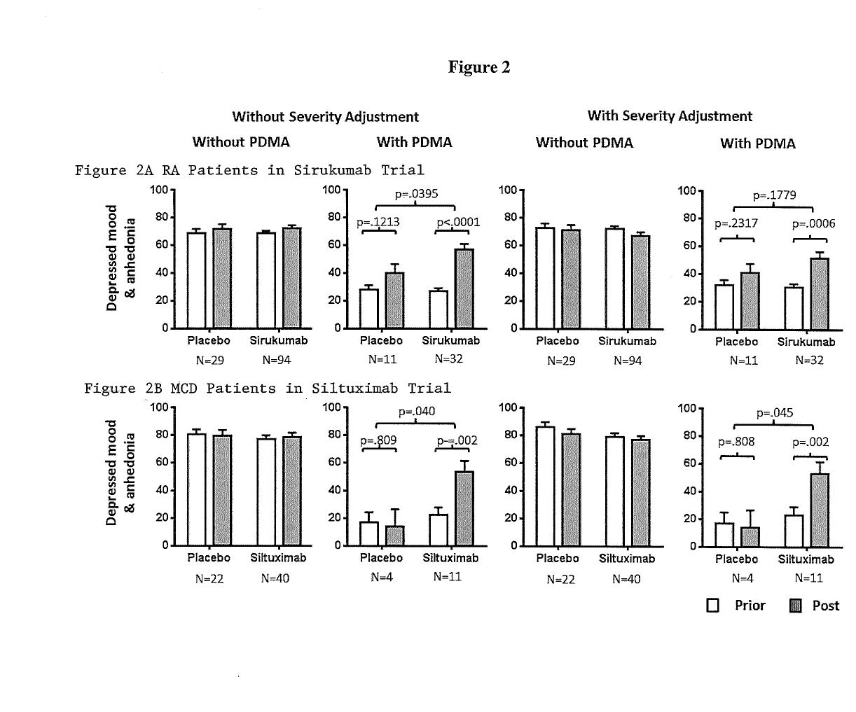

In RA patients with PDMA, the depressed-mood and anhedonia ratings significantly improved under sirukumab, and this improvement was significant both with and without adjustment for baseline RA severity is detected in the sirukumab; the corresponding changes in depression symptoms in the placebo arm were not significant (FIG. 2A). The mood improvement was significantly greater in the sirukumab arm than in placebo arm without, but not with, adjustment for DAS28CRP severity (FIG. 2A). Sirukumab also significantly improved ratings of depressed mood and anhedonia in RA non-responders as defined by the ACR50 (FIG. 3A). Neither sirukumab nor placebo significantly altered mood measures in patients without PDMA at baseline (potentially due to ceiling effects; FIG. 2A).

In MCD patients with PDMA, significant improvement in depressed-mood and anhedonia ratings was observed in patients with and without correction for MCDOS severity in the siltuximab, but not the placebo arm (FIG. 2B). The mood improvement was significantly greater in the siltuximab arm than in the placebo arm without, but not with, adjustment for MCDOS severity (FIG. 2B). Neither siltuximab nor placebo significantly altered depressive symptom ratings in patients without PDMA at baseline (potentially due to ceiling effects; FIG. 2B).

### Example 5: Effect of IL-6 Neutralizing Antibodies on Measures of Fatigue

In the sirukumab study, RA patients with PDMA at trial entry exhibit significantly greater improvement on the fatigue measure in the sirukumab than the placebo arm. Adjusting for DAS28-CRP severity, only the within sirukumab treatment effect remains significant (FIG. 3A). In patients without PDMA at trial entry, fatigue improved significantly from baseline to week 12 in both the treated and placebo group (FIG. 3A).

In the siltuximab study, MCD patients with PDMA at trial entry exhibit significantly greater improvement on the fatigue measure in the siltuximab than the placebo arm. Adjusting for MCDOS severity, only the within siltuximab treatment effect remains significant (FIG. 3B). In patients without PDMA at baseline, siltuximab treatment significantly improved the fatigue score without, but not with, adjustment for RA severity (FIG. 3B).

Fatigue is an overlapping symptom of sickness and major depressive syndromes. The treatment outcomes on fatigue appeared similar to those of depressed mood and anhedonia (FIGS. 3A and 3B). The improvement in fatigue under IL-6 antibody administration was observed in patients considered non-responders with respect to the primary disease severity measured with DAS28 and MCDOS.

### Example 6: Effect of IL-6 Neutralizing Antibodies on Measures of Depressed Mood, Anhedonia and Fatigue in the Responders and Non-Responder of Primary Diseases

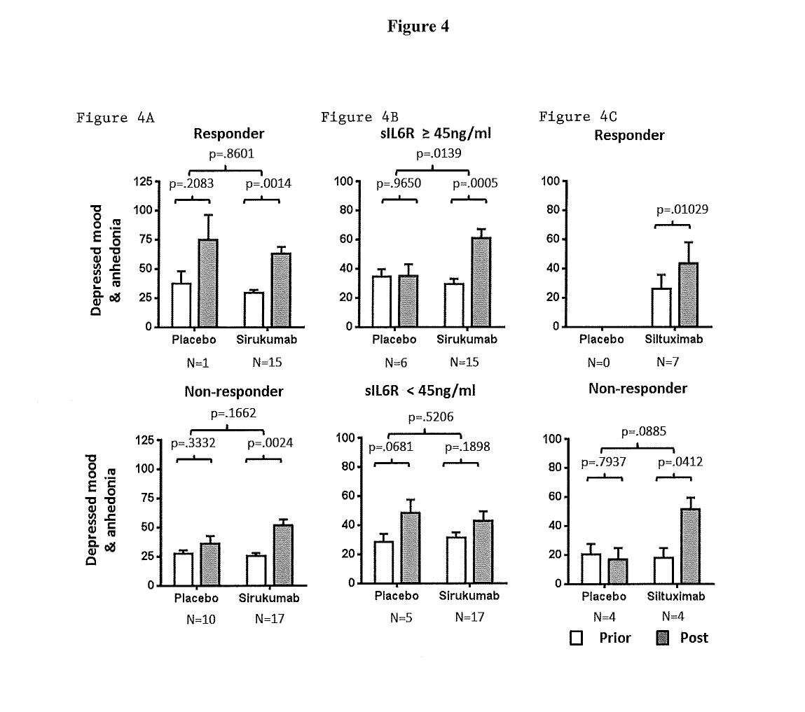

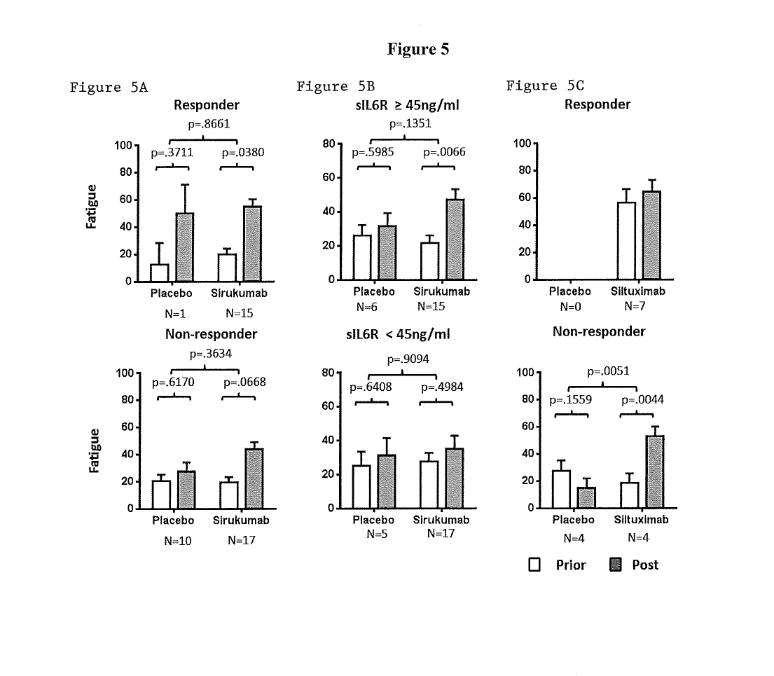

In RA patients with PDMA prior to the treatment, significant improvement in depressed mood and anhedonia was detected in sirukumab treated subjects regardless whether being classified as RA responders (ACR50) or non-responders (FIG. 4A). In contrast, sirukumab treatment resulted in significant improvement on the fatigue measure in RA responders, but not non-responders (FIG. 5A).

In MCD patients with PDMA, there were no responders in the placebo group. In the cohort of MCD non-responders, the sample size is small with only 4 subjects in each group. Nevertheless, siltuximab treatment significantly improved both depressed mood and anhedonia (FIG. 4C), and fatigue (FIG. 5C) in MCD non-responders.

The fact that both sirukumab and siltuximab treatment significantly improved mood measures in the RA and MCD non-responders (FIG. 4A, C) suggested that the effects of these drugs on depressed mood and anhedonia could be partially independent or at least synergistic

### Example 7: Baseline Serum Biomarkers and Clinical Improvement

In RA patients with PDMA, no significant correlation was found between change in depressed mood and anhedonia and pre-treatment levels of CRP, IL-6 and sgp130 in the sirukumab arm (Table 13). Improvement in depressed mood and anhedonia was significantly correlated with baseline sIL-6R level (p=0.015; Table 13). Further stratification of PDMA subjects into a ‘high’ versus ‘low’ group based on the median split value of sIL-6R (>45 ng/mL) revealed enrichment in patients whose mood improved when treated with sirukumab (FIG. 4B). In MCD patients with PDMA, no significant correlation between mood improvement on treatment with siltuximab and baseline levels of CRP or IL-6 were detected (Table 12). sIL-6R and sgp130 were not measured in the MCD trial.

TABLE 13
Relationships between reductions of depression scores and levels
of some biomarkers at trial entry in patients with PDMA

| | Sirukumab | Placebo | Siltuximab | Placebo |
|---|---|---|---|---|
| | Depression Score | Depression Score | Depression Score | Depression Score |
| | Week 12-Week 0 | Week 12-Week 0 | Week 6-Week 0 | Week 6-Week 0 |
| | N = 32 | N = 11 | N = 11 | N = 4 |
| | Spearman r (p) | Spearman r (p) | Spearman r (p) | Spearman r (p) |
| CRP | −0.23 (0.21) | 0.01 (0.98) | −0.38 (0.25) | 0.11 (0.89) |
| IL-6 | −0.13 (0.49) | 0.003 (0.99) | −0.33 (0.36) | 0.63 (0.37) |
| sIL-6R | 0.44 (0.015) | −0.12 (0.75) | Not measured | Not measured |
| sgp130 | 0.27 (0.15) | 0.32 (0.40) | Not measured | Not measured |

1. A method for treating depression or fatigue in a subject comprising administering to the subject an effective amount of a pharmaceutical composition comprising an agent that blocks binding of IL-6 to IL-6 receptor.

2. The method of claim 1 wherein the subject has depressed mood, fatigue, or anhedonia.

3. The method of claim 1 wherein the subject has rheumatoid arthritis.

4. The method of claim 1 wherein the subject has multicentric Castleman's disease.

5. The method of claim 1 wherein the agent that blocks binding of IL-6 to IL-6 receptor comprises an isolated antibody or an antigen-binding fragment thereof.

6. The method according to claim 5 wherein the isolated antibody or an antigen-binding fragment thereof comprises the following CDR's:
i) CDRH1 as set out in SEQ ID NO. 135; and
ii) CDRH2 as set out in SEQ ID NO. 136; and
iii) CDRH3 as set out in SEQ ID NO. 137; and
iv) CDRL1 as set out in SEQ ID NO. 132; and
v) CDRL2 as set out in SEQ ID NO. 133; and
vi) CDRL3 as set out in SEQ ID NO. 134; and wherein
X₁ is A or G, X₂ is S or R, X₃ is H, I, S, or Y, X₄ is S or Y, X₅ is S or F, X₆ is F, L, M, or T, X₇ is N or E, X₈ is A or T, X₉ is M, C, S or Q, X₁₀ is Q or C, X₁₁ is T or Q, X₁₂ is F, S, or T, X₁₃ is S or P, X₁₄ is L or M, X₁₅ is A or I, X₁₆ is S or P, X₁₇ is Y or IV, X₁₈ is T, E, or Y, X₁₉ is Y or F, X₂₀ is P, S, D, or Y, X₂₁ is V or D, X₂₂ is T or A, X₂₃ is G or P, X₂₄ is S, Y, T, or N, and X₂₅ is Y, T, F, or I.

7. The method of claim 5 wherein the isolated antibody or an antigen-binding fragment thereof comprises a heavy chain variable regions and a light chain variable regions of SEQ ID NO:99 and SEQ ID NO:97 respectively.

8. The method of claim 5 wherein the isolated antibody or an antigen-binding fragment thereof comprises a heavy chain variable region and a light chain variable regions of SEQ ID NO:139 and SEQ ID NO:140 respectively.

9. The method of claim 5 wherein the isolated antibody or an antigen-binding fragment thereof comprises a heavy chain CDR1 comprising the amino acid sequence of SEQ ID NO:141, a heavy chain CDR2 comprising the amino acid sequence of SEQ ID NO:142, a heavy chain CDR3 comprising the amino acid sequence of SEQ ID NO:143, a light chain CDR1 comprising the amino acid sequence of SEQ ID NO:144, a light chain CDR2 comprising the amino acid sequence of SEQ ID NO:145, a light chain CDR3 comprising the amino acid sequence of SEQ ID NO:146.

10. The method of claim 5 wherein the isolated antibody or an antigen-binding fragment thereof is administered at a dose of 25-100 mg every 2-4 weeks.

11. The method of claim 10 wherein the isolated antibody or an antigen-binding fragment thereof is administered at a dose selected from the group comprising 100 mg every 2 weeks, 25 mg every 4 weeks, 50 mg every 4 weeks, and 100 mg every 4 weeks.

12. The method of claim 5 wherein the isolated antibody or an antigen-binding fragment thereof is administered at a dose of 11 mg/kg every 3 weeks.

13. The method of claim 5 wherein the isolated antibody or an antigen-binding fragment thereof is administered subcutaneously.

14. The method of claim 5 wherein the isolated antibody or an antigen-binding fragment thereof is administered intravenously.

15. A method for determining responsiveness of an individual having depression to the treatment with IL-6 antibody or a fragment thereof, comprising:
a. measuring the amount of soluble IL-6 receptor (sIL-6R) in a biological sample from the subject;
b. providing a threshold value correlating the amount of sIL-6R and responsiveness;
c. comparing the amount of sIL-6R to the threshold value; wherein responsiveness is determined when the amount of sIL-6R exceeds the threshold value and/or wherein a lack of responsiveness is determined when the amount of sIL-6R does not exceed the threshold value; and
d. treating the individual with an agent that blocks binding of IL-6 to IL-6 receptor.

16. The method of claim 15 wherein the threshold value is 45 ng/mL.

17. The method of claim 15 wherein the biological sample is serum.

18. The method of claim 15 wherein the patient has rheumatoid arthritis or multicentric Castleman's disease.

19. The method of claim 15 wherein the IL-6 antibody or a fragment thereof comprises a heavy chain variable regions and a light chain variable regions of SEQ ID NO:99 and SEQ ID NO:97, respectively.

20. The method of claim 15 wherein the IL-6 antibody or a fragment thereof comprises a heavy chain variable regions and a light chain variable regions of SEQ ID NO:139 and SEQ ID NO:140, respectively.


SEQUENCE LISTING (146 sequences)

SEQ ID NO: 1 - protein, 10 aa, Homo sapiens
SASHSVSYMY

SEQ ID NO: 2 - protein, 30 aa, Homo sapiens
AGTGCCAGCCATAGTGTAAGTTACATGTAC

SEQ ID NO: 3 - protein, 10 aa, Homo sapiens
SASISVSYMY

SEQ ID NO: 4 - protein, 30 aa, Homo sapiens
AGTGCCAGCATTAGTGTAAGTTACATGTAC

SEQ ID NO: 5 - protein, 10 aa, Homo sapeins
SARSSVSYMY

SEQ ID NO: 6 - protein, 30 aa, Homo sapiens
AGTGCCCGGTCAAGTGTAAGTTACATGTAC

SEQ ID NO: 7 - protein, 10 aa, Homo sapiens
SASYSVSYMY

SEQ ID NO: 8 - protein, 30 aa, Homo sapiens
AGTGCCAGCTATAGTGTAAGTTACATGTAC

SEQ ID NO: 9 - protein, 10 aa, Homo sapiens
SASSSVFYMY

SEQ ID NO: 10 - protein, 30 aa, Homo sapiens
AGTGCCAGCTCAAGTGTATTTTACATGTAC

SEQ ID NO: 11 - protein, 10 aa, Homo sapiens
SGSSYVSYMY

SEQ ID NO: 12 - protein, 30 aa, Homo sapiens
AGTGGCAGCTCATATGTAAGTTACATGTAC

SEQ ID NO: 13 - protein, 10 aa, Homo sapiens
SALSSVSYMY

SEQ ID NO: 14 - protein, 30 aa, Homo sapiens
AGTGCCCTGTCAAGTGTAAGTTACATGTAC

SEQ ID NO: 15 - protein, 10 aa, Homo sapiens
SASSSVSYMY

SEQ ID NO: 16 - protein, 30 aa, Homo sapiens
AGTGCCAGCTCAAGTGTAAGTTACATGTAC

SEQ ID NO: 17 - protein, 7 aa, Homo sapiens
DFSNLAS

SEQ ID NO: 18 - protein, 21 aa, Homo sapiens
GACTTTTCCAACCTGGCTTCT

SEQ ID NO: 19 - protein, 7 aa, Homo sapiens
DLSNLAS

SEQ ID NO: 20 - protein, 21 aa, Homo sapiens
GACCTGTCCAACCTGGCTTCT

SEQ ID NO: 21 - protein, 7 aa, Homo sapiens
DMSNLAS

SEQ ID NO: 22 - protein, 21 aa, Homo sapiens
GACATGTCCAACCTGGCTTCT

SEQ ID NO: 23 - protein, 7 aa, Homo sapiens
DTSNLTS

SEQ ID NO: 24 - protein, 21 aa, Homo sapiens
GACACATCCAACCTGACGTCT

SEQ ID NO: 25 - protein, 7 aa, Homo sapiens
DTSELAS

SEQ ID NO: 26 - protein, 21 aa, Homo sapiens
GACACATCCGAGCTGGCTTCT

SEQ ID NO: 27 - protein, 7 aa, Homo sapiens
DTSNLAS

SEQ ID NO: 28 - protein, 21 aa, Homo sapiens
GACACATCCAACCTGGCTTCT

SEQ ID NO: 29 - protein, 9 aa, Homo sapiens
MQWSGYPYT

SEQ ID NO: 30 - protein, 27 aa, Homo sapiens
ATGCAGTGGAGTGGTTACCCATACACG

SEQ ID NO: 31 - protein, 9 aa, Homo sapiens
CQWSGYPYT

SEQ ID NO: 32 - protein, 27 aa, Homo sapiens
TGTCAGTGGAGTGGTTACCCATACACG

SEQ ID NO: 33 - protein, 9 aa, Homo sapiens
SCWSGYPYT

SEQ ID NO: 34 - protein, 27 aa, Homo sapiens
TCTGTGTGGAGTGGTTACCCATACACG

SEQ ID NO: 35 - protein, 9 aa, Homo sapiens
SQWSGYPYT

SEQ ID NO: 36 - protein, 27 aa, Homo sapiens
TCTCAGTGGAGTGGTTACCCATACACG

SEQ ID NO: 37 - protein, 10 aa, Homo sapiens
GFTFSSFALS

SEQ ID NO: 38 - protein, 30 aa, Homo sapiens
GGATTCACCTTTAGTAGCTTTGCCCTTTCT

SEQ ID NO: 39 - protein, 10 aa, Homo sapiens
GFTFSPFAMS

SEQ ID NO: 40 - protein, 30 aa, Homo sapiens
GGATTCACCTTTAGTCCTTTTGCCATGTCT

SEQ ID NO: 41 - protein, 10 aa, Homo sapiens
GFQFSSFAMS

SEQ ID NO: 42 - protein, 30 aa, Homo sapiens
GGATTCCAGTTTAGTAGCTTTGCCATGTCT

SEQ ID NO: 43 - protein, 10 aa, Homo sapiens
GFTTSSFAMS

SEQ ID NO: 44 - protein, 30 aa, Homo sapiens
GGATTCACCACTAGTAGCTTTGCCATGTCT

SEQ ID NO: 45 - protein, 10 aa, Homo sapiens
GFQFSPFAMS

SEQ ID NO: 46 - protein, 30 aa, Homo sapiens
GGATTCCAGTTTAGTCCTTTTGCCATGTCT

SEQ ID NO: 47 - protein, 10 aa, Homo sapiens
GFTFSSFAMS

SEQ ID NO: 48 - protein, 30 aa, Homo sapiens
GGATTCACCTTTAGTAGCTTTGCCATGTCT

SEQ ID NO: 49 - protein, 17 aa, Homo sapiens
KASSGGSYTYYPDTVTG

SEQ ID NO: 50 - protein, 51 aa, Homo sapiens
AAAGCGAGTAGTGGTGGGAGTTACACCTACTATCCTGACACTGTGACGGGC

SEQ ID NO: 51 - protein, 17 aa, Homo sapiens
KISSGGSYEYYPDTVTG

SEQ ID NO: 52 - protein, 51 aa, Homo sapiens
AAAATTAGTAGTGGTGGGAGTTACGAGTACTATCCTGACACTGTGACGGGC

SEQ ID NO: 53 - protein, 17 aa, Homo sapiens
KISSGGSYYYYPDTVTG

SEQ ID NO: 54 - protein, 51 aa, Homo sapiens
AAAATTAGTAGTGGTGGGAGTTACTATTACTATCCTGACACTGTGACGGGC

SEQ ID NO: 55 - protein, 17 aa, Homo sapiens
KISSGGSWTYYPDTVTG

SEQ ID NO: 56 - protein, 51 aa, Homo sapiens
AAAATTAGTAGTGGTGGGAGTTGGACCTACTATCCTGACACTGTGACGGGC

SEQ ID NO: 57 - protein, 17 aa, Homo sapiens
KISPGGSYTYYPDTVTG

SEQ ID NO: 58 - protein, 51 aa, Homo sapiens
AAAATTAGTCCGGGTGGGAGTTACACCTACTATCCTGACACTGTGACGGGC

SEQ ID NO: 59 - protein, 17 aa, Homo sapiens
KISPGGSWTYYSDTVTG

SEQ ID NO: 60 - protein, 51 aa, Homo sapiens
AAAATTAGTCCGGGTGGGAGTTGGACCTACTATTCTGACACTGTGACGGGC

SEQ ID NO: 61 - protein, 17 aa, Homo sapiens
KISSGGSYTYYPDTVTG

SEQ ID NO: 62 - protein, 51 aa, Homo sapiens
AAAATTAGTAGTGGTGGGAGTTACACCTACTATCCTGACACTGTGACGGGC

SEQ ID NO: 63 - protein, 17 aa, Homo sapiens
KISSGGSYTYFPDTVTG

SEQ ID NO: 64 - protein, 51 aa, Homo sapiens
AAAATTAGTAGTGGTGGGAGTTACACCTACTTTCCTGACACTGTGACGGGC

SEQ ID NO: 65 - protein, 17 aa, Homo sapiens
KISSGGSYTYYPDTVAG

SEQ ID NO: 66 - protein, 51 aa, Homo sapiens
AAAATTAGTAGTGGTGGGAGTTACACCTACTATCCTGACACTGTGGCTGGC

SEQ ID NO: 67 - protein, 17 aa, Homo sapiens
KISSGGSYTYYDDTVTG

SEQ ID NO: 68 - protein, 51 aa, Homo sapiens
AAAATTAGTAGTGGTGGGAGTTACACCTACTATGATGACACTGTGACGGGC

SEQ ID NO: 69 - protein, 17 aa, Homo sapiens
KISSGGSYTYYSDTVTG

SEQ ID NO: 70 - protein, 51 aa, Homo sapiens
AAAATTAGTAGTGGTGGGAGTTACACCTACTATTCTGACACTGTGACGGGC

SEQ ID NO: 71 - protein, 17 aa, Homo sapiens
KISSGGSYTYYPDTVTP

SEQ ID NO: 72 - protein, 51 aa, Homo sapiens
AAAATTAGTAGTGGTGGGAGTTACACCTACTATCCTGACACTGTGACGCCG

SEQ ID NO: 73 - protein, 17 aa, Homo sapiens
KISSGGSYTYYPDTDTG

SEQ ID NO: 74 - protein, 51 aa, Homo sapiens
AAAATTAGTAGTGGTGGGAGTTACACCTACTATCCTGACACTGATACGGGC

SEQ ID NO: 75 - protein, 17 aa, Homo sapiens
KISPGGSYTYYSDTVTG

SEQ ID NO: 76 - protein, 51 aa, Homo sapiens
AAAATTAGTCCGGGTGGGAGTTACACCTACTATTCTGACACTGTGACGGGC

SEQ ID NO: 77 - protein, 17 aa, Homo sapiens
KISPGGSWTYYDDTVTG

SEQ ID NO: 78 - protein, 51 aa, Homo sapiens
AAAATTAGTCCGGGTGGGAGTTGGACCTACTATGATGACACTGTGACGGGC

SEQ ID NO: 79 - protein, 10 aa, Homo sapiens
QLWGSYALDY

SEQ ID NO: 80 - protein, 30 aa, Homo sapiens
CAGTTATGGGGGTCGTATGCTCTTGACTAC

SEQ ID NO: 81 - protein, 10 aa, Homo sapiens
QLWGYYALDT

SEQ ID NO: 82 - protein, 30 aa, Homo sapiens
CAGTTATGGGGGTACTATGCTCTTGACACG

SEQ ID NO: 83 - protein, 10 aa, Homo sapiens
QLWGTYALDY

SEQ ID NO: 84 - protein, 30 aa, Homo sapiens
CAGTTATGGGGGACTTATGCTCTTGACTAC

SEQ ID NO: 85 - protein, 10 aa, Homo sapiens
QLWGNYALDY

SEQ ID NO: 86 - protein, 30 aa, Homo sapiens
CAGTTATGGGGGAATTATGCTCTTGACTAC

SEQ ID NO: 87 - protein, 10 aa, Homo sapiens
QLWGYYALDF

SEQ ID NO: 88 - protein, 30 aa, Homo sapiens
CAGTTATGGGGGTACTATGCTCTTGACTTT

SEQ ID NO: 89 - protein, 10 aa, Homo sapiens
QLWGYYALDI

SEQ ID NO: 90 - protein, 30 aa, Homo sapiens
CAGTTATGGGGGTACTATGCTCTTGACATT

SEQ ID NO: 91 - protein, 10 aa, Homo sapiens
QLWGYYALDY

SEQ ID NO: 92 - protein, 30 aa, Homo sapiens
CAGTTATGGGGGTACTATGCTCTTGACTAC

SEQ ID NO: 93 - protein, 106 aa, Homo sapiens
EIVLTQSPATLSLSPGERATLSCSASSSVSYMYWYQQKPGQAPRLLIYDTSNLASGIPARFSGSGSGTDFTLTISSLEPEDFAVYYCSQWSGYPYTFGGGTKVEIK

SEQ ID NO: 94 - DNA, 318 bp, Homo sapiens
gaaattgtgttgacacagtctccagccaccctgtctttgtctccaggggaaagagccaccctctcctgcagtgccagctcaagtgtaagttacatgtactggtaccaacagaaacctggccaggctcccaggctcctcatctatgacacatccaacctggcttctggcatcccagccaggttcagtggcagtgggtctgggacagacttcactctcaccatcagcagcctagagcctgaagattttgcagtttattactgttctcagtggagtggttacccatacacgttcggcggagggaccaaggtggagatcaaa

SEQ ID NO: 95 - protein, 119 aa, Homo sapiens
EVQLVESGGGLVQPGGSLRLSCAASGFTFSSFAMSWVRQAPGKGLEWVAKISSGGSYTYYPDTVTGRFTISRDNAKNSLYLQMNSLRAEDTAVYYCARQLWGYYALDYWGQGTTVTVSS

SEQ ID NO: 96 - DNA, 357 bp, Homo sapiens
gaggtgcagctggtggagtctgggggaggcttggtccagcctggggggtccctgagactctcctgtgcagcctctggattcacctttagtagctttgccatgtcttgggtccgccaggctccagggaaggggctggagtgggtggccaaaattagtagtggtgggagttacacctactatcctgacactgtgacgggccgattcaccatctccagagacaacgccaagaactcactgtatctgcaaatgaacagcctgagagccgaggacacggctgtgtattactgtgcgagacagttatgggggtactatgctcttgactactggggccaagggaccacggtcaccgtctcctca

SEQ ID NO: 97 - protein, 106 aa, Homo sapiens
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 98 - DNA, 318 bp, Homo sapiens
gaaattgtgttgacacagtctccagccaccctgtctttgtctccaggggaaagagccaccctctcctgcagtgccagcattagtgtaagttacatgtactggtaccaacagaaacctggccaggctcccaggctcctcatctatgacatgtccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 99 - protein, 119 aa, Homo sapiens
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 100 - DNA, 357 bp, Homo sapiens
gaggtgcagctggtggagtctgggggaggcttggtccagcctggggggtccctgagactctcctgtgcagcctctggattcacctttagtccttttgccatgtcttgggtccgccaggctccagggaaggggctggagtgggtggccaaaattagtccgggtgggagttggacctactattctgacactgtgacgggccgattcaccatctccagagacaacgccaagaactcactgtatctgcaaatgaacagcctgagagccgaggacacggctgtgtattactgtgcgagacagttatgggggtactatgctcttgacatttggggccaagggaccacggtcaccgtctcctca

SEQ ID NO: 101 - protein, 90 aa, Homo sapiens
EIVLTQSPATLSLSPGERATLSCSASYSVSYMYWYQQKPGQAPRLLIYDMSNLASGIPARFSGSGSGTDFTLTISSLEPEDFAVYYCMQW

SEQ ID NO: 102 - DNA, 318 bp, Homo sapiens
gaaattgtgttgacacagtctccagccaccctgtctttgtctccaggggaaagagccaccctctcctgcagtgccagctatagtgtaagttacatgtactggtaccaacagaaacctggccaggctcccaggctcctcatctatgacatgtccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 103 - protein, 119 aa, Homo sapiens
EVQLVESGGGLVQPGGSLRLSCAASGFQFSSFAMSWVRQAPGKGLEWVAKISPGGSYTYYSDTVTGRFTISRDNAKNSLYLQMNSLRAEDTAVYYCARQLWGYYALDFWGQGTTVTVSS

SEQ ID NO: 104 - DNA, 357 bp, Homo sapiens
gaggtgcagctggtggagtctgggggaggcttggtccagcctggggggtccctgagactctcctgtgcagcctctggattccagtttagtagctttgccatgtcttgggtccgccaggctccagggaaggggctggagtgggtggccaaaattagtccgggtgggagttacacctactattctgacactgtgacgggccgattcaccatctccagagacaacgccaagaactcactgtatctgcaaatgaacagcctgagagccgaggacacggctgtgtattactgtgcgagacagttatgggggtactatgctcttgacttttggggccaagggaccacggtcaccgtctcctca

SEQ ID NO: 105 - protein, 23 aa, Homo sapiens
EIVLTQSPATLSLSPGERATLSC

SEQ ID NO: 106 - protein, 15 aa, Homo sapiens
WYQQKPGQAPRLLIY

SEQ ID NO: 107 - protein, 32 aa, Homo sapiens
GIPARFSGSGSGTDFTLTISSLEPEDFAVYYC

SEQ ID NO: 108 - protein, 10 aa, Homo sapiens
FGGGTKVEIK

SEQ ID NO: 109 - protein, 25 aa, Homo sapiens [sequence omitted]

SEQ ID NO: 110 - protein, 14 aa, Homo sapiens
WVRQAPGKGLEWVA

SEQ ID NO: 111 - protein, 32 aa, Homo sapiens
RFTISRDNAKNSLYLQMNSLRAEDTAVYYCAR

SEQ ID NO: 112 - protein, 11 aa, Homo sapiens
WGQGTTVTVSS

SEQ ID NO: 113 - protein, 17 aa, Homo sapiens
EISSGGSYTYYPDTVTG

SEQ ID NO: 114 - protein, 10 aa, Homo sapiens
GLWGYYALDY

SEQ ID NO: 115 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 116 - protein, 106 aa, Homo sapiens; identical to SEQ ID NO: 93

SEQ ID NO: 117 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagctcaagtgtaagttacatgtactggtaccaacagaaacctggccaggctcccaggctcctcatctatgacacatccaacctggcttctggcatcccagccaggttcagtggcagtgggtctgggacagacttcactctcaccatcagcagcctagagcctgaagattttgcagtttattactgttctcagtggagtggttacccatacacgttcggcggagggaccaaggtggagatcaaa

SEQ ID NO: 118 - protein, 119 aa, Homo sapiens
EVQLVESGGGLVQPGGSLRLSCAASGFTFSSFAMSWVRQAPGKGLEWVAKISPGGSYTYYPDTVTGRFTISRDNAKNSLYLQMNSLRAEDTAVYYCARQLWGYYALDYWGQGTTVTVSS

SEQ ID NO: 119 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattcacctttagtagctttgccatgtcttgggtccgccaggctccagggaaggggctggagtgggtggccaaaattagtcccggtgggagttacacctactatcctgacactgtgacgggccgattcaccatctccagagacaacgccaagaactcactgtatctgcaaatgaacagcctgagagccgaggacacggctgtgtattactgtgcgagacagttatgggggtactatgctcttgactactggggccaagggaccacggtcaccgtctcctca

SEQ ID NO: 120 - protein, 106 aa, Homo sapiens
EIVLTQSPATLSLSPGERATLSCSASSSVSYMYWYQQKPGQAPRLLIYDFSNLASGIPARFSGSGSGTDFTLTISSLEPEDFAVYYCMQWSGYPYTFGGGTKVEIK

SEQ ID NO: 121 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagctcaagtgtaagttacatgtactggtaccaacagaaacctggccaggctcccaggctcctcatctatgacttctccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 122 - protein, 119 aa, Homo sapiens
EVQLVESGGGLVQPGGSLRLSCAASGFQFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 123 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattccagtttagtccctttgccatgtcttgggtccgccaggctccagggaaggggctggagtgggtggccaaaattagtcccggtgggagttggacctactatagcgacactgtgacgggccgattcaccatctccagagacaacgccaagaactcactgtatctgcaaatgaacagcctgagagccgaggacacggctgtgtattactgtgcgagacagttatgggggtactatgctcttgacatttggggccaagggaccacggtcaccgtctcctca

SEQ ID NO: 124 - protein, 106 aa, Homo sapiens; identical to SEQ ID NO: 97

SEQ ID NO: 125 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagcattagtgtaagttacatgtactggtaccaacagaaacctggccaggctcccaggctcctcatctatgacatgtccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 126 - protein, 119 aa, Homo sapiens
EVQLVESGGGLVQPGGSLRLSCAASGFQFSSFAMSWVRQAPGKGLEWVAKISPGGSYTYYSDTVTGRFTISRDNAKNSLYLQMNSLRAEDTAVYYCARQLWGYYALDIWGQGTTVTVSS

SEQ ID NO: 127 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattccagtttagtagctttgccatgtcttgggtccgccaggctccagggaaggggctggagtgggtggccaaaattagtcccggtgggagttacacctactatagcgacactgtgacgggccgattcaccatctccagagacaacgccaagaactcactgtatctgcaaatgaacagcctgagagccgaggacacggctgtgtattactgtgcgagacagttatgggggtactatgctcttgacatttggggccaagggaccacggtcaccgtctcctca

SEQ ID NO: 128 - protein, 106 aa, Homo sapiens
EIVLTQSPATLSLSPGERATLSCSASYSVSYMYWYQQKPGQAPRLLIYDFSNLASGIPARFSGSGSGTDFTLTISSLEPEDFAVYYCMQWSGYPYTFGGGTKVEIK

SEQ ID NO: 129 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagctacagtgtaagttacatgtactggtaccaacagaaacctggccaggctcccaggctcctcatctatgacttctccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 130 - protein, 119 aa, Homo sapiens
EVQLVESGGGLVQPGGSLRLSCAASGFQFSPFAMSWVRQAPGKGLEWVAKISPGGSWTYYPDTDTGRFTISRDNAKNSLYLQMNSLRAEDTAVYYCARQLWGYYALDFWGQGTTVTVSS

SEQ ID NO: 131 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattccagtttagtccctttgccatgtcttgggtccgccaggctccagggaaggggctggagtgggtggccaaaattagtcccggtgggagttggacctactatcctgacactgacacgggccgattcaccatctccagagacaacgccaagaactcactgtatctgcaaatgaacagcctgagagccgaggacacggctgtgtattactgtgcgagacagttatgggggtactatgctcttgacttctggggccaagggaccacggtcaccgtctcctca

SEQ ID NO: 132 - protein, 10 aa, Homo sapiens
  misc_feature (2)..(5): Xaa can be any naturally occurring amino acid
  misc_feature (7)..(7): Xaa can be any naturally occurring amino acid
SXXXXVXYMY

SEQ ID NO: 133 - protein, 7 aa, Homo sapiens
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
DXSXLXS

SEQ ID NO: 134 - protein, 9 aa, Homo sapiens
  misc_feature (1)..(2): Xaa can be any naturally occurring amino acid
XXWSGYPYT

SEQ ID NO: 135 - protein, 10 aa, Homo sapiens
  misc_feature (3)..(4): Xaa can be any naturally occurring amino acid
  misc_feature (6)..(6): Xaa can be any naturally occurring amino acid
  misc_feature (9)..(9): Xaa can be any naturally occurring amino acid
GFXXSXFAXS

SEQ ID NO: 136 - protein, 17 aa, Homo sapiens
  misc_feature (2)..(2): Xaa can be any naturally occurring amino acid
  misc_feature (4)..(4): Xaa can be any naturally occurring amino acid
  misc_feature (8)..(9): Xaa can be any naturally occurring amino acid
  misc_feature (11)..(12): Xaa can be any naturally occurring amino acid
  misc_feature (15)..(17): Xaa can be any naturally occurring amino acid
KXSXGGSXXYXXDTXXX

SEQ ID NO: 137 - protein, 10 aa, Homo sapiens
  misc_feature (5)..(5): Xaa can be any naturally occurring amino acid
  misc_feature (10)..(10): Xaa can be any naturally occurring amino acid
QLWGXYALDX

SEQ ID NO: 138 - protein, 9 aa, Homo sapiens
QQWSGYPYT

SEQ ID NO: 139 - protein, 119 aa, Mus musculus [sequence omitted]

SEQ ID NO: 140 - protein, 106 aa, Mus musculus [sequence omitted]

SEQ ID NO: 141 - protein, 5 aa, Mus musculus
SFAMS

SEQ ID NO: 142 - protein, 17 aa, Mus musculus
EISSGGSYTYYPDTVTG

SEQ ID NO: 143 - protein, 10 aa, Mus musculus
GLWGYYALDY

SEQ ID NO: 144 - protein, 10 aa, Mus musculus
SASSSVSYMY

SEQ ID NO: 145 - protein, 7 aa, Mus musculus
DTSNLAS

SEQ ID NO: 146 - protein, 9 aa, Mus musculus
QQWSGYPYT